SEQUENCE LISTING (11 sequences)

SEQ ID NO: 1 - DNA, 1155 bp, Homo sapiens
atggaacttcataacctgagctctccatctccctctctctcctcctctgttctccctccctccttctctccctcaccctcctctgctccctctgcctttaccactgtgggggggtcctctggagggccctgccaccccacctcttcctcgctggtgtctgccttcctggcaccaatcctggccctggagtttgtcctgggcctggtggggaacagtttggccctcttcatcttctgcatccacacgcggccctggacctccaacacggtgttcctggtcagcctggtggccgctgacttcctcctgatcagcaacctgcccctccgcgtggactactacctcctccatgagacctggcgctttggggctgctgcctgcaaagtcaacctcttcatgctgtccaccaaccgcacggccagcgttgtcttcctcacagccatcgcactcaaccgctacctgaaggtggtgcagccccaccacgtgctgagccgtgcttccgtgggggcagctgcccgggtggccgggggactctgggtgggcatcctgctcctcaacgggcacctgctcctgagcaccttctccggcccctcctgcctcagctacagggtgggcacgaagccctcggcctcgctccgctggcaccaggcactgtacctgctggagttcttcctgccactggcgctcatcctctttgctattgtgagcattgggctcaccatccggaaccgtggtctgggcgggcaggcaggcccgcagagggccatgcgtgtgctggccatggtggtggccgtctacaccatctgcttcttgcccagcatcatctttggcatggcttccatggtggctttctggctgtccgcctgccgatccctggacctctgcacacagctcttccatggctccctggccttcacctacctcaacagtgtcctggaccccgtgctctactgcttctctagccccaacttcctccaccagagccgggccttgctgggcctcacgcggggccggcagggcccagtgagcgacgagagctcctaccaaccctccaggcagtggcgctaccgggaggcctctaggaaggcggaggccatagggaagctgaaagtgcagggcgaggtctctctggaaaaggaaggctcctcccagggctga

SEQ ID NO: 2 - protein, 384 aa, Homo sapiens
MELHNLSSPSPSLSSSVLPPSFSPSPSSAPSAFTTVGGSSGGPCHPTSSSLVSAFLAPILALEFVLGLVGNSLALFIFCIHTRPWTSNTVFLVSLVAADFLLISNLPLRVDYYLLHETWRFGAAACKVNLFMLSTNRTASVVFLTAIALNRYLKVVQPHHVLSRASVGAAARVAGGLWVGILLLNGHLLLSTFSGPSCLSYRVGTKPSASLRWHQALYLLEFFLPLALILFAIVSIGLTIRNRGLGGQAGPQRAMRVLAMVVAVYTICFLPSIIFGMASMVAFWLSACRSLDLCTQLFHGSLAFTYLNSVLDPVLYCFSSPNFLHQSRALLGLTRGRQGPVSDESSYQPSRQWRYREASRKAEAIGKLKVQGEVSLEKEGSSQG

SEQ ID NO: 3 - protein, 387 aa, Homo sapiens
MNRHHLQDHFLEIDKKNCCVFRDDFIAKVLPPVLGLEFIFGLLGNGLALWIFCFHLKSWKSSRIFLFNLAVADFLLIICLPFVMDYYVRRSDWNFGDIPCRLVLFMFAMNRQGSIIFLTVVAVDRYFRVVHPHHALNKISNWTAAIISCLLWGITVGLTVHLLKKKLLIQNGPANVCISFSICHTFRWHEAMFLLEFLLPLGIILFCSARIIWSLRQRQMDRHAKIKRAITFIMVVAIVFVICFLPSVVVRIRIFWLLHTSGTQNCEVYRSVDLAFFITLSFTYMNSMLDPVVYYFSSPSFPNFFSTLINRCLQRKMTGEPDNNRSTSVELTGDPNKTRGAPEALMANSGEPWSPSYLGPTSNNHSKKGHCHQEPASLEKQLGCCIE

SEQ ID NO: 4 - protein, 319 aa, Homo sapiens
MPFPNCSAPSTVVATAVGVLLGLECGLGLLGNAVALWTFLFRVRVWKPYAVYLLNLALADLLLAACLPFLAAFYLSLQAWHLGRVGCWALRFLLDLSRSVGMAFLAAVALDRYLRVVHPRLKVNLLSPQAALGVSGLVWLLMVALTCPGLLISEAAQNSTRCHSFYSRADGSFSIIWQEALSCLQFVLPFGLIVFCNAGIIRALQKRLREPEKQPKLQRAQALVTLVVVLFALCFLPCFLARVLMHIFQNLGSCRALCAVAHTSDVTGSLTYLHSVVNPVVYCFSSPTFRSSYRRVFHTLRGKGQAAEPPDFNPRDSYS

SEQ ID NO: 5 - DNA, 24 bp, Artificial
  Primer
cagcccatggaacttcataacctg

SEQ ID NO: 6 - DNA, 23 bp, Artificial
  Primer
ctggccctcagccctgggaggag

SEQ ID NO: 7 - DNA, 20 bp, Artificial
  Primer
gctgcctgcaaagtcaacct

SEQ ID NO: 8 - DNA, 18 bp, Artificial
  Primer
tggctgtgaggaagacaa

SEQ ID NO: 9 - DNA, 18 bp, Artificial
  Probe
ccaccaaccgcacggcaa

SEQ ID NO: 10 - DNA, 17 bp, Artificial
  Primer
ctcctgatcagcaacct

SEQ ID NO: 11 - DNA, 20 bp, Artificial
  Primer
ttggtggacagcatgaagag

(19) United States
(12) Patent Application Publication
(10) Pub. No.: US 2003/0186873 A1
(43) Pub. Date: Oct. 2, 2003

(54) Nucleic acid encoding a G-protein-coupled receptor, and uses thereof

(76) Inventors: Haifeng Eishingdrelo, Montville, NJ (US); Jidong Cai, Whippany, NJ (US); Anthony Sandrasagra, Princeton, NJ (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/348,083

(22) Filed: Jan. 21, 2003

Related U.S. Application Data
(60) Provisional application No. 60/351,006, filed on Jan. 23, 2002.

(30) Foreign Application Priority Data
May 9, 2002 (GB) 0210597.1

(51) Int. Cl. A61K 38/17; C12Q 1/68; C07H 21/04; C12P 21/02; C12N 5/06; C07K 14/705

(52) U.S. Cl. 514/12; 530/350; 530/388.22; 536/23.5; 435/6; 435/69.1; 435/320.1; 435/325

(57) ABSTRACT

Provided herein is a novel and useful G-protein coupled receptor that is involved in signal transduction with respect to inflammation and physiological immunological response. Also provided are methods of using the receptor to screen for molecules that may modulate the activity of the receptor. Such molecules may readily have applications in treating a plethora of inflammation and immunologically related diseases and disorders.

### PRIORITY CLAIM

This application claims priority under 35 U.S.C. 119 from U.S. Ser. No. 60/351,006 filed on Jan. 23, 2002 and British Application No. 0210597.1 filed on May 9, 2001, wherein said applications are hereby incorporated by reference herein in their entireties.

### FIELD OF THE INVENTION

The present invention relates generally to a novel nucleic acid molecule that encodes for GAVE6, a heretofore unknown G-protein-coupled receptor, along with uses of the nucleic acid molecule and GAVE6.

### BACKGROUND OF THE INVENTION

The G protein-coupled receptors (GPCRs) are a large family of integral membrane proteins that are involved in cellular signal transduction. GPCRs respond to a variety of extracellular signals, including neurotransmitters, hormones, odorants and light, and are capable of transducing signals so as to initiate a second messenger response within the cell. Many therapeutic drugs target GPCRs because those receptors mediate a wide variety of physiological responses, including inflammation, vasodilation, heart rate, bronchodilation, endocrine secretion and peristalsis.

GPCRs are characterized by extracellular domains, seven transmembrane domains and intracellular domains. Some of the functions the receptors perform, such as binding ligands and interacting with G proteins, are related to the presence of certain amino acids in critical positions. For example, a variety of studies have shown that differences in amino acid sequence in GPCRs account for differences in affinity to either a natural ligand or a small molecule agonist or antagonist. In other words, minor differences in sequence can account for different binding affinities and activities. (See, for example, Meng et al., J Bio Chem (1996) 271(50):32016-20; Burd et al., J Bio Chem (1998) 273(51):34488-95; and Hurley et al., J Neurochem (1999) 72(1):413-21). In particular, studies have shown that amino acid sequence differences in the third intracellular domain can result in different activities. Myburgh et al. found that alanine 261 of intracellular loop 3 of gonadotropin releasing hormone receptor is crucial for G protein coupling and receptor internalization (Biochem J (1998) 331(Part 3):893-6). Wonerow et al. studied the thyrotropin receptor and demonstrated that deletions in the third intracellular loop resulted in constitutive receptor activity (J Bio Chem (1998)273(14):7900-5).

In general, the action of the binding of an endogenous ligand to a receptor results in a change in the conformation of the intracellular domain(s) of the receptor allowing for coupling between the intracellular domain(s) and an intracellular component, a G-protein. Several G proteins exist, such as G_{q}, Gₛ, Gᵢ, G_{z} and Gₒ (see, e.g. Dessauer et al., Clin Sci (Colch) (1996) 91(5):527-37). The IC-3 loop as well as the carboxy terminus of the receptor interact with the G proteins (Pauwels et al., Mol Neurobiol (1998) 17(1-3):109-135 and Wonerow et. al., supra). Some GPCRs are “promiscuous” with respect to G proteins, i.e., a GPCR can interact with more than one G protein (see, e.g., Kenakin, Life Sciences (1988)43:1095).

Ligand activated GPCR coupling with G protein begins a signaling cascade process (referred to as “signal transduction”). Such signal transduction ultimately results in cellular activation or cellular inhibition.

GPCRs exist in the cell membrane in equilibrium between two different conformations: an “inactive” and an “active” state. A receptor in an inactive state is unable to link to the intracellular signaling transduction pathway to produce a biological response (exceptions exist, such as during over-expression of receptor in transduced cells, see e.g., www.creighton.edu/Pharmacology/inverse.htm.). Modulation of the conformation to the active state allows linkage to the transduction pathway (via the G protein) and produces a biological response. Agonists bind and make the active conformation much more likely. However, sometimes, if there is already a considerable response in the absence of any agonist, such receptors are said to be constitutively active (i.e., already in an active conformation or ligand independent or autonomous active state). When agonists are added to such systems, an enhanced response routinely is observed. However, when a classical antagonist is added, binding by such molecules produces no effect. On the other hand, some antagonists cause an inhibition of the constitutive activity of the receptor, suggesting that the latter class of drugs technically are not antagonists but are agonists with negative intrinsic activity. Those drugs are called inverse agonists, www.creighton.edu/Pharmacology/inverse.htm.).

Traditional study of receptors has proceeded from the assumption that the endogenous ligand first be identified before discovery could move forward to identify antagonists and other receptor effector molecules. Even where antagonists might have been discovered first, the dogmatic response was to identify the endogenous ligand (WO 00/22131). However, as the active state is the most useful for assay screening purposes, obtaining such constitutive receptors, especially GPCRs, would allow for the facile isolation of agonists, partial, agonists, inverse agonists and antagonists in the absence of information concerning endogenous ligands. Moreover, in diseases that result from disorders of receptor activity, drugs that cause inhibition of constitutive activity, or more specifically, reduce the effective activated receptor concentration, could be discovered more readily by assays using receptors in the autonomous active state. For example, as receptors that may be transfected into patients to treat disease, the activity of such receptors may be fine-tuned with inverse agonists discovered by such assays.

Diseases such as asthma, chronic obstructive pulmonary disease (COPD) and rheumatoid arthritis (RA) generally are considered to have an inflammatory etiology involving T helper cells, monocyte-macrophages and eosinophils. Current anti-inflammatory therapy with corticosteroids is effective in asthma but is associated with metabolic and endocrine side effects. The same is possibly true for inhaled formulations that can be absorbed through lung or nasal mucosa. Satisfactory oral therapies for RA or COPD currently are lacking.

Eosinophils mediate much of the airway dysfunction in allergy and asthma. Interleukin-5 (IL-5) is an eosinophil growth and activating cytokine. Studies have shown IL-5 to be necessary for tissue eosinophilia and for eosinophil-mediated tissue damage resulting in airway hyperresponsiveness (Chang et al., J Allergy Clin Immunol (1996) 98(5 pt 1):922-931 and Duez et al., Am J Respir Crit Care Med (2000) 161(1):200-206). IL-5 is made by T-helper-2 cells (Th2) following allergen (e.g. house dust mite antigen) exposure in atopic asthma.

RA is believed to result from accumulation of activated macrophages in the affected synovium. Interferon γ(IFNγ) is a T-helper-1 (Th1) cell-derived cytokine with numerous proinflammatory properties. It is the most potent macrophage activating cytokine and induces MHC class II gene transcription contributing to a dendritic cell-like phenotype.

Lipopolysaccharide (LPS) is a component of gram-negative bacterial cell walls that elicits inflammatory responses, including tumor necrosis factor α(TNFα) release. The efficacy of intravenous anti-TNFα therapy in RA has been demonstrated in the clinic. COPD is thought also to result from macrophage accumulation in the lung, the macrophages produce neutrophil chemoattractants (e.g., IL-8: de Boer et al., J Pathol (2000) 190(5):619-626). Both macrophages and neutrophils release cathepsins that cause degradation of the alveolar wall. It is believed that lung epithelium can be an important source for inflammatory cell chemoattractants and other inflammatory cell-activating agents (see, for example, Thomas et al., J Virol (2000) 74(18):8425-8433; Lamkhioued et al., Am J Respir Crit Care Med (2000) 162(2 Pt. 1):723-732; and Sekiya et al., J Immunol (2000) 165(4):2205-2213).

Given the role GPCRs have in disease and the ability to treat diseases by modulating the activity of GPCRs, identification and characterization of previously unknown GPCRs can provide for the development of new compositions and methods for treating disease states that involve the activity of a GPCR. Accordingly, what is needed is the discovery, isolation and characterization of novel and useful nucleic acid molecules that encode for heretofore unknown GPCRs.

What is also needed are assays that utilize such heretofore unknown GPCRs to identify molecules that can serve potential agonists or antagonists of particular GPCRS. These molecules may readily have applications as therapeutic agents for modulating the activity of GPCRs in vivo, and thus, treat a plethora of diseases related to GPCR activity.

The citation of any reference herein should not be construed as an admission that such reference is available as “Prior Art” to the instant application.

### SUMMARY OF THE INVENTION

The instant invention identifies and characterizes the expression of a novel constitutively active GPCR, GAVE6, and provides compositions and methods for applying the discovery to the identification and treatment of related diseases.

Thus broadly, the present invention extends to an isolated nucleic acid molecule comprising a DNA sequence of FIG. 1 (SEQ ID NO: 1), a variant thereof, a fragment thereof, or an analog or a derivative thereof. Such a variant of the present invention may be an allelic variant, a degenerate variant, or an allelic variant that results in a degenerate change in the sequence.

Moreover, the present invention extends to an isolated nucleic acid molecule hybridizable to the isolated nucleic acid molecule of SEQ ID NO: 1, or a variant thereof, under stringent hybridization conditions. Yet further, the present invention extends to an isolated nucleic acid molecule hybridizable to a nucleic acid molecule that is complementary to the DNA sequence of SEQ ID NO: 1 under stringent hybridization conditions. Stringent hybridization conditions are described infra.

Furthermore, the present invention extends to an isolated nucleic acid molecule comprising a DNA sequence that encodes a polypeptide comprising an amino acid sequence of SEQ ID NO: 2.

Optionally, an isolated nucleic acid molecule of the present invention as described above may be detectably labeled. Examples of detectable labels having applications herein include, but certainly are not limited to an enzyme, a radioactive isotope, or a chemical which fluoresces. Particular examples of detectable labels are described infra.

Particular polypeptides are also encompassed within the present invention. For example, the present invention extends to a purified polypeptide comprising the amino acid sequence of SEQ ID NO: 2, a conservative variant thereof, or an analog or derivative thereof. Optionally, a polypeptide of the present invention may be detectably labeled.

In addition, the present invention extends to antibodies wherein a polypeptide of the present invention is the immunogen used in production of the antibodies. These antibodies can be monoclonal or polyclonal. Moreover, the antibodies can be “chimeric” as, for example, they may comprise protein domains of antibodies raised against a purified polypeptide of the present invention in different species. In a particular embodiment, an antibody of the present invention may be “humanized.” Naturally, an antibody of the present invention may be detectable labeled. Particular examples of detectable labels having applications herein are described infra.

The present invention further extends to an expression vector comprising a nucleic acid molecule comprising a DNA sequence of SEQ ID NO: 1, a variant thereof, an analog or derivative thereof, or a fragment thereof, operatively associated with an expression control element. Furthermore, an expression vector of the present invention may comprise an isolated nucleic acid molecule hybridizable under stringent hybridization conditions to an isolated nucleic acid molecule comprising a DNA sequence of SEQ ID NO: 1, operatively associated with an expression control element, or is hybridizable under stringent hybridization conditions to a hybridization probe that is complementary to an isolated nucleic acid molecule comprising a DNA sequence of SEQ ID NO: 1, wherein the hybridization probe is operatively associated with an expression control element. A particular example of an expression control element having applications herein is a promoter. Examples of particular promoters applicable to the present invention, include, but are not limited to early promoters of hCMV, early promoters of SV40, early promoters of adenovirus, early promoters of vaccinia, early promoters of polyoma, late promoters of SV40, late promoters of adenovirus, late promoters of vaccinia, late promoters of polyoma, the lac the trp system, the TAC system, the TRC system, the major operator and promoter regions of phage lambda, control regions of fd coat protein, 3-phosphoglycerate kinase promoter, acid phosphatase promoter, or promoters of yeast α mating factor.

With an expression vector of the present invention, one may transfect or transform a host cell and produce a polypeptide comprising an amino acid sequence of SEQ ID NO: 2, or a variant thereof The host cell may be either a prokaryotic cell or a eukaryotic cell. Particular examples of unicellular hosts having applications herein include E. coli, Pseudonomas, Bacillus, Strepomyces, yeast, CHO, R1.1, B-W, L-M, COS1, COS7, BSC1, BSC40, BMT10 and Sf9 cells, to name only a few.

Moreover, the present invention further extends to a method for producing a purified polypeptide comprising the amino acid sequence of SEQ ID NO: 2, a variant thereof, or a fragment thereof. Such a method comprises culturing a host cell transformed or transfected with an expression vector of the present invention under conditions that provide for expression of the purified polypeptide, and then recovering the purified polypeptide from the unicellular host, the culture surrounding the host cell, or from both.

Moreover, the present invention extends to assays for identifying compounds that can modulate the activity of GAVE6. Such compounds can be an agonist, an antagonist, or an inverse agonist of GAVE6. Hence accordingly, the present invention extends to a method for identifying an agonist of GAVE6 comprising contacting a potential agonist with a cell expressing GAVE6 in the presence of an endogenous ligand, and determining whether the signaling activity of GAVE6 is increased when the potential agonist is present, relative to the signaling activity of GAVE6 in the absence of the potential agonist.

Likewise, the present invention extends to a method for identifying an inverse agonist of GAVE6. Such a method comprises contacting a potential inverse agonist with a cell expressing GAVE6, and determining whether the signaling activity of GAVE6 in the presence of the potential inverse agonist and an endogenous ligand or agonist is decreased relative to the signaling activity of GAVE6 under conditions in which the presence of an endogenous ligand or agonist, but in absence of potential inverse agonist, and is decreased in the presence of an endogenous ligand or agonist.

Naturally, the present invention extends to methods for identifying an antagonist of GAVE6. Such a method comprises the steps of contacting a potential antagonist with a cell expressing GAVE6, and determining whether in the presence of said potential antagonist the signaling activity of GAVE6 is decreased relative to the activity of GAVE6 in the presence of an endogenous ligand or agonist.

Accordingly, it is an object of the present invention to provide an isolated nucleic acid sequence which encodes a GAVE6 protein, a fragment thereof, or a variant thereof.

It is also an object of the present invention to provide a variant of an nucleic acid molecule comprising a DNA sequence of SEQ ID NO: 1, or is hybridizable to SEQ ID NO: 1 under stringent conditions.

It is a further object of the present invention to provide an amino acid sequence for GAVE6, along with variant thereof, a fragment thereof, or an analog or derivative thereof.

It is a further object of the present invention to provide an expression vector comprising a DNA sequence that encodes GAVE6, a variant thereof, a fragment thereof, or an analog or derivative thereof, wherein the DNA sequence is operably associated with an expression control element.

It is still a further object of the present invention to provide an antibody having GAVE6, an variant thereof, an analog or derivative thereof, or a fragment thereof, as an immunogen.

Yet another object of the present invention involves methods for identify compounds that can modulate the activity of GAVE6 protein. Such modulator may be an antagonist of GAVE6, an agonist of GAVE6, or inverse agonist of GAVE6.

It is a still further object of the present invention to provide pharmaceutical compositions for use in modulating GAVE6 activity. Such modulation can be used to treat a variety of diseases related to GAVE6 activity, e.g., various inflammatory diseases, asthma, chronic obstructive pulmonary disease (COPD), and rheumatoid arthritis, to name only a few.

These and other aspects of the present invention will be better appreciated by reference to the following drawings and Detailed Description.

### BRIEF DESCRIPTION OF THE DRAWINGS

FIG. 1: DNA sequence that encodes GAVE6 (SEQ ID NO: 1).

FIG. 2: Amino acid sequence of GAVE6 (SEQ ID NO: 2)

FIG. 3: Comparison of the amino acid sequence of GAVE6 (SEQ ID NO: 2) with the amino acid sequences of HM74 and GPR31 (SEQ ID NO: 3 and SEQ ID NO: 4, respectively).

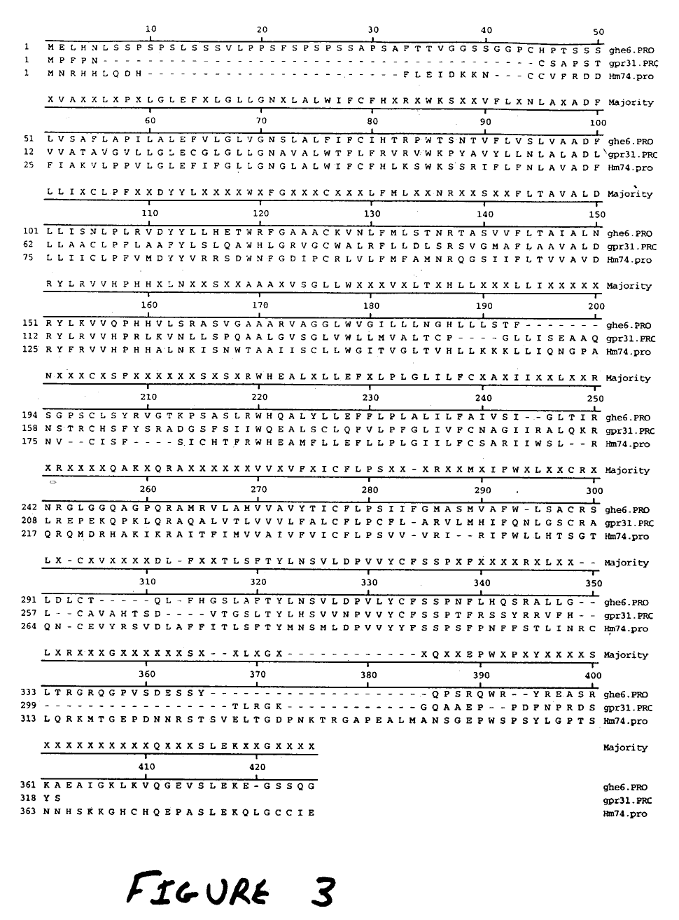

FIG. 4: Northern Blot of the transcription of GAVE6 in various tissues.

FIG. 5: GAVE6 Expression Profile in various tissues in a human organ/tissue panel.

### DETAILED DESCRIPTION OF THE INVENTION

As explained above, the present invention relates to the surprising and unexpected discovery of a heretofore unknown nucleic acid molecule that encodes a heretofore unknown G protein-coupled receptor referred to herein as GAVE6. In particular, it has been discovered that GAVE6 is expressed in immune tissues or organs, such as the kidney, liver and small intestine.

Various terms and phrases used throughout the instant Specification and Claims to describe the present invention are set forth below:

As used herein, the term “modulator” refers to a moiety (e.g., but not limited to a ligand and a candidate compound) that modulates the activity of GAVE6. A modulator of the present invention may be an agonist, a partial agonist, an antagonist, or an inverse agonist of GAVE6.

As used herein, the term “agonist” refers to moieties (e.g., but not limited to ligands and candidate compounds) that activate the intracellular response when bound to the receptor, or enhance GTP binding to membranes.

As used herein, the term “partial agonist” refers to moieties (e.g., but not limited to ligands and candidate compounds) that activate the intracellular response when bound to the receptor to a lesser degree/extent than do agonists, or enhance GTP binding to membranes to a lesser degree/extent than do agonists.

As used herein, the term “antagonist” refers moieties (e.g., but not limited to ligands and candidate compounds) that competitively bind to the receptor at the same site as does an agonist. However, an antagonist does not activate the intracellular response initiated by the active form of the receptor and thereby can inhibit the intracellular responses by agonists or partial agonists. In a related aspect, antagonists do not diminish the baseline intracellular response in the absence of an agonist or partial agonist.

As used herein, the term “inverse agonist” refers to moieties (e.g., but not limited to ligand and candidate compound) that bind to a constitutively active receptor and inhibit the baseline intracellular response. The baseline response is initiated by the active form of the receptor below the normal base level of activity that is observed in the absence of agonists or partial agonists, or decrease of GTP binding to membranes.

As used herein, the term “candidate compound” refers to a moiety (e.g., but not limited to a chemical compound) that is amenable to a screening technique. In one embodiment, the term does not include compounds that were publicly known to be compounds selected from the group consisting of agonist, partial agonist, inverse agonist or antagonist of GAVE6. Those compounds were identified by traditional drug discovery processes involving identification of an endogenous ligand specific for a receptor, and/or screening of candidate compounds against a receptor wherein such a screening requires a competitive assay to assess efficacy.

As used herein, the terms “constitutively activated receptor” or “autonomously active receptor,” are used herein interchangeably, and refer to a receptor subject to activation in the absence of ligand. Such constitutively active receptors can be endogenous (e.g., GAVE6) or non-endogenous; i.e., GPCRs can be modified by recombinant means to produce mutant constitutive forms of wild-type GPCRs (e.g., see EP 1071701; WO 00/22129; WO 00/22131; and U.S. Pat. Nos. 6,150,393 and 6,140,509 which are hereby incorporated by reference herein in their entireties.

As used herein, the term “constitutive receptor activation” refers to the stabilization of a receptor in the active state by means other than binding of the receptor with the endogenous ligand or chemical equivalent thereof.

As used herein, the term “ligand” refers to a moiety that binds to another molecule, wherein the moiety includes, but certainly is not limited to a hormone or a neurotransmitter, and further, wherein the moiety stereoselectively binds to a receptor.

As used herein, the term “family,” when referring to a protein or a nucleic acid molecule of the invention, is intended to mean two or more proteins or nucleic acid molecules having a seemingly common structural domain and having sufficient amino acid or nucleotide sequence identity as defined herein. Such family members can be naturally occurring and can be from either the same or different species. For example, a family can contain a first protein of human origin and a homologue of that protein of murine origin, as well as a second, distinct protein of human origin and a murine homologue of that second protein. Members of a family also may have common functional characteristics.

As used herein interchangeably, the terms “GAVE6 activity”, “biological activity of GAVE6” and “functional activity of GAVE6”, refer to an activity exerted by a GAVE6 protein, polypeptide or nucleic acid molecule on a GAVE6 responsive cell as determined in vivo or in vitro, according to standard techniques. A GAVE6 activity can be a direct activity, such as an association with or an enzymatic activity on a second protein or an indirect activity, such as a cellular signaling activity mediated by interaction of the GAVE6 protein with a second protein. In a particular embodiment, a GAVE6 activity includes, but is not limited to at least one or more of the following activities: (i) the ability to interact with proteins in the GAVE6 signaling pathway; (ii) the ability to interact with a GAVE6 ligand; and (iii) the ability to interact with an intracellular target protein.

Furthermore, in accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning. A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage or cosmid, to name only a few, to which another DNA segment may be attached so as to bring about the replication of the attached segment. A “replicon” is any genetic element (e.g., plasmid, chromosome, virus) that functions as an autonomous unit of DNA replication in vivo, i.e., capable of replication under its own control. Particular examples of vectors are described infra.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

“Homologous recombination” refers to the insertion of a foreign DNA sequence of a vector in a chromosome. In particular, the vector targets a specific chromosomal site for homolo- gous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Isolated Nucleic Acid Molecules of the Present Invention

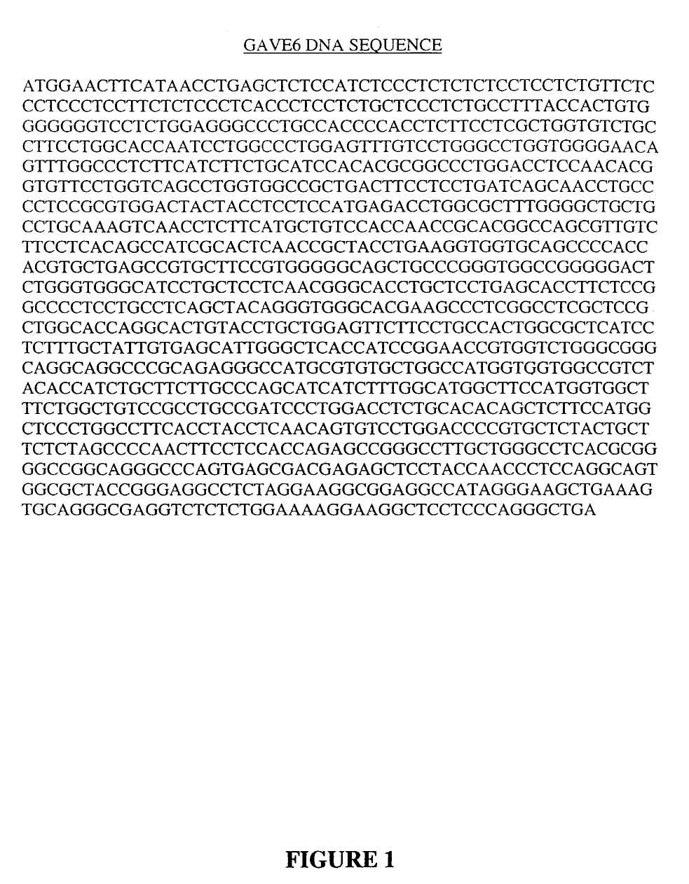

In one aspect, the present invention extends to an isolated nucleic acid molecule comprising DNA sequence of FIG. 1 (SEQ ID NO: 1), a variant thereof, a fragment thereof, or an analog or derivative thereof.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

An “isolated” nucleic acid molecule is one that is separated from other nucleic acid molecules present in the natural source of the nucleic acid. In particular, an “isolated” nucleic acid is free of sequences that naturally flank the nucleic acid encoding GAVE6 (i.e., sequences located at the 5′ and 3′ ends of the nucleic acid) in the genomic DNA of the organism from which the nucleic acid is derived. In various embodiments, the isolated GAVE6 nucleic acid molecule can contain less than about 5 kb, 4 kb, 3 kb, 2 kb, 1 kb, 0.5 kb or 0.1 kb of nucleotide sequences that naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques or substantially free of chemical precursors or other chemicals when synthesized chemically.

A nucleic acid molecule of the present invention, e.g., a nucleic acid molecule having the nucleotide sequence of SEQ ID NO: 1 or a fragment or complement of any of that nucleotide sequence, or an analog or derivative thereof, can be isolated using standard molecular biology techniques and the sequence information provided herein. Using all or a portion of the nucleic acid sequence of SEQ ID NO: 1 as a hybridization probe, GAVE6 nucleic acid molecules can be isolated using standard hybridization and cloning techniques (e.g., as described in Sambrook et al).

A nucleic acid molecule of the invention can be amplified using cDNA, mRNA or genomic DNA as a template and appropriate oligonucleotide primers according to standard PCR amplification techniques. Such primers may be readily made using information set forth in SEQ ID NO: 1, and routine laboratory techniques. The nucleic acid so amplified can be cloned into an appropriate vector and characterized by DNA sequence analysis. Furthermore, oligonucleotides corresponding to GAVE6 nucleotide sequences can be prepared by standard synthetic techniques, e.g., using an automated DNA synthesizer.

Isolated Nucleic Acid Molecule Hybridizable to GAVE6 DNA

The present invention further extends to isolated nucleic acid molecules hybridizable to GAVE6 DNA, hybridizable to a hybridization probe that is complementary under stringent hybridization conditions GAVE6 DNA, or hybridizable under stringent hybridization conditions to both. In particular, the present invention extends to an isolated nucleic acid molecule that is hybridizable under stringent hybridization conditions to a nucleic acid molecule comprising a DNA sequence of SEQ ID NO: 1, or to a probe that is complementary to an isolated nucleic acid molecule comprising a DNA sequence of SEQ ID NO: 1.

A nucleic acid molecule is “hybridizable” to another nucleic acid molecule, such as a cDNA, genomic DNA, or RNA, when a single stranded form of the nucleic acid molecule can anneal to another nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ, have been derived (see Sambrook et al., supra, 9.50-0.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid molecule is at least about 20 nucleotides; particularly at least about 30 nucleotides; more particularly at least about 40 nucleotides, even more particularly about 50 nucleotides, and yet more particularly at least about 60 nucleotides. In a particular embodiment of the present invention, a hybridizable nucleic acid molecule of the invention is at least 300, 325, 350, 375, 400, 425, 450, 500, 550, 600, 650, 700, 800, 900, 1000 or 1100 nucleotides in length and hybridizes under stringent conditions to the nucleic acid molecule comprising the nucleotide sequence, preferably the coding sequence, of SEQ ID NO: 1 a complement thereof, or a fragment thereof.

As used herein, the term “hybridizes under stringent conditions” is intended to describe conditions for hybridization and washing under which nucleotide sequences at least 55%, 60%, 65%, 70% and preferably 75% or more complementary to each other typically remain hybridized. Such stringent conditions are known to those skilled in the art and can be found in “Current Protocols in Molecular Biology”, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of SEQ ID NO: 1 or the complement thereof corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein). The skilled artisan will appreciate that the conditions may be modified in view of sequence-specific variables,(e.g., length, G-C richness etc.).

The invention contemplates encompassing nucleic acid fragments of GAVE6 that are diagnostic of GAVE6-like molecules that have similar properties. The diagnostic fragments can arise from any portion of the GAVE6 gene including flanking sequences. The fragments can be used as probe of a library practicing known methods.

Moreover, a nucleic acid molecule of the invention can comprise only a portion of a nucleic acid sequence encoding GAVE6, for example, a fragment that can be used as a probe or primer, or a fragment encoding a biologically active portion of GAVE6. For example, such a fragment can comprise, but is not limited to, a region encoding amino acid residues about 1 to about 14 of SEQ ID NO: 2. The nucleotide sequence determined from the cloning of the human GAVE6 gene allows for the generation of probes and primers for identifying and/or cloning GAVE6 homologues in other cell types, e.g., from other tissues, as well as GAVE6 homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NO: 1 or of a naturally occurring mutant of SEQ ID NO: 1. Probes based on the human GAVE6 nucleotide sequence can be used to detect transcripts or genomic sequences encoding the similar or identical proteins.

As used herein, the terms “fragment” or “portion” of an isolated nucleic acid molecule of the present invention comprise at least 12, particularly about 25, more particularly about 50, 75, 100, 125, 150, 175, 200, 250, 300, 350 or 400 consecutive nucleotides. Consequently, a “fragment” of an isolated nucleic acid molecule of the present invention is not merely 1 or 2 nucleotides.

Similarly, a “fragment” or “portion” of a polypeptide of the present invention comprises at least 9 contiguous amino acid residues. A particular example of a fragment of a polypeptide of the present invention comprises is an epitope to which a GAVE6 antibody, or fragment thereof, binds.

A nucleic acid fragment encoding a “biologically active portion of GAVE6” can be prepared by isolating a portion of SEQ ID NO: 1 that encodes a polypeptide having a GAVE6 biological activity, expressing the encoded portion of GAVE6 protein (e.g., by recombinant expression in vitro) and assessing the activity of the encoded portion of GAVE6. The invention further encompasses nucleic acid molecules that differ from the nucleotide sequence of SEQ ID NO:l due to degeneracy of the genetic code, and thus encode the same GAVE6 protein as that encoded by the nucleotide sequence shown in SEQ ID NO: 1.

Homologous Nucleic Acid Molecules

The present invention further extends to an isolated nucleic acid molecule that is homologous to a GAVE6 DNA molecule, e.g., is homologous to an isolated nucleic acid molecule having a DNA sequence of SEQ ID NO: 1. Two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks using default parameters, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra. Moreover, nucleic acid molecules encoding GAVE6 proteins from other species (GAVE6 homologues) with a nucleotide sequence that differs from that of a human GAVE6, are intended to be within the scope of the invention.

Variants of an Isolated Nucleic Acid Molecule of the Present Invention

The present invention further extends to variants of an isolated nucleic acid molecule comprising a DNA sequence of SEQ ID NO: 1. Such variants can be degenerate, allelic, or a combination thereof.

Nucleic acid molecules corresponding to natural allelic variants and homologues of the GAVE6 cDNA of the invention can be isolated based on identity with the human GAVE6 nucleic acids disclosed herein using the human cDNA or a portion thereof, as a hybridization probe according to standard hybridization techniques under stringent hybridization conditions.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

Moreover, due to degenerate nature of codons in the genetic code, a GAVE6 protein of the present invention can be encoded by numerous isolated nucleic acid molecules. “Degenerate nature” refers to the use of different three-letter codons to specify a particular amino acid pursuant to the genetic code. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC |
| | or CUA or CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG |
| | or AGU or AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or GAG |
| Asparagine (Asn or N) | AAU or AAC |
| Lysine (Lys or K) | AAA or AAG |
| Aspartic Acid (Asp or D) | GAU or GAG |
| Glutamic Acid (Glu or E) | GAA or GAG |
| Cysteine (Cys or C) | UGU or UGC |
| Arginine (Arg or R) | CGU or CGC or CGA or CGG |
| | or AGA or AGG |
| Glycine (Gly or G) | GGU or GGC or GGA or GGG |
| Tryptophan (Trp or W) | UGG |
| Termination codon | UAA (ochre) or UAG (amber) |
| | or UGA (opal) |

It should be understood that the codons specified above are for RNA sequences. The corresponding codons for DNA have a T substituted for U.

In addition to the human GAVE6 nucleotide sequence shown in SEQ ID NO: 1, it will be appreciated by those skilled in the art that DNA sequence polymorphisms that lead to changes in the amino acid sequences of GAVE6 may exist within a population (e.g., the human population). Such genetic polymorphism in the GAVE6 gene may exist among individuals within a population due to natural allelic variation. An allele is one of a group of genes that occur alternatively at a given genetic locus. As used herein, the terms “gene” and “recombinant gene” refer to nucleic acid molecules comprising an open reading frame encoding a GAVE6 protein, preferably a mammalian GAVE6 protein. As used herein, the phrase “allelic variant” refers to a nucleotide sequence that occurs at a GAVE6 locus or to a polypeptide encoded by the nucleotide sequence. Alternative alleles can be identified by sequencing the gene of interest in a number of different individuals. That can be carried out readily by using hybridization probes to identify the same genetic locus in a variety of individuals. Any and all such nucleotide variations and resulting amino acid polymorphisms or variations in GAVE6 that are the result of natural allelic variation and that do not alter the functional activity of GAVE6 are intended to be within the scope of the invention.

Moreover, variants of an isolated nucleic acid molecule of the present invention can be readily made by one of ordinary skill in the art using routine laboratory techniques, e.g., site-directed mutagenesis.

Antisense Nucleotide Sequences

The instant invention also extends to antisense nucleic acid molecules, i.e., molecules that are complementary to a sense nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire GAVE6 coding strand or to only a portion thereof, e.g., all or part of the protein coding region (or open reading frame). An antisense nucleic acid molecule can be antisense to a noncoding region of the coding strand of a nucleotide sequence encoding GAVE6. The noncoding regions (“5′ and 3′ untranslated regions”) are the 5′ and 3′ sequences that flank the coding region and are not translated into amino acids.

Given the coding strand sequences encoding GAVE6 disclosed herein (e.g., SEQ ID NO: 1), antisense nucleic acids of the invention can be designed according to the rules of Watson & Crick base pairing. The antisense nucleic acid molecule can be complementary to the entire coding region of GAVE6 mRNA, but more preferably is an oligonucleotide that is antisense to only a portion of the coding or noncoding region of GAVE6 mRNA. For example, the antisense oligonucleotide can be complementary to the region surrounding the translation start site of GAVE6 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be synthesized chemically using naturally occurring nucleotides or various chemically modified nucleotides designed to increase the biological stability of the molecules, or to increase the physical stability of the duplex formed between the antisense and sense nucleic acids, e.g., phosphorothioate derivatives, phosphonate derivatives and acridine-substituted nucleotides can be used.

Examples of modified nucleotides that can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-adenine, 7-methylguanine, 5-methylaminomethyluracil, 5-methoxyaminomethyl-2-thiouracil, β-D-mannosylqueosine, 5-methoxycarboxymethyluracil, 5-methoxyuracil, 2-methylthio-N⁶-isopentenyladenine, uracil-5-oxyacetic acid, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl)uracil and 2,6-diaminopurine. Alternatively, the antisense nucleic acid can be produced biologically using an expression vector into that a nucleic acid has been subcloned in an antisense orientation (i.e., RNA transcribed from the inserted nucleic acid will be of an antisense orientation to a target nucleic acid of interest).

The antisense nucleic acid molecules of the invention typically are administered to a subject or generated in situ so as to hybridize with or bind to cellular mRNA and/or genomic DNA encoding a GAVE6 protein thereby to inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix, or to a regulatory region of GAVE6.

An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that the molecules specifically bind to receptors or antigens expressed on a selected cell surface, e.g., by linking the antisense nucleic acid molecules to peptides or antibodies that bind to cell surface receptors or antigens. The antisense nucleic acid molecules also can be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in that the strands run parallel to each other (Gaultier et al., Nucleic Acids Res (1987)15:6625-6641). The antisense nucleic acid molecule also can comprise a methylribonucleotide (Inoue et al., Nucleic Acids Res (1987) 15:6131-6148) or a chimeric RNA-DNA analogue (Inoue et al., FEBS Lett (1987) 215:327-330).

Ribozymes

The invention also encompasses ribozymes. Ribozymes are catalytic RNA molecules with ribonuclease activity that are capable of cleaving a single-stranded nucleic acid, such as an mRNA, that hybridizes to the ribozyme. Thus, ribozymes (e.g., hammerhead ribozymes (described in Haselhoff et al., Nature (1988) 334:585-591)) can be used to cleave catalytically GAVE6 mRNA transcripts, and thus inhibit translation of GAVE6 mRNA. A ribozyme having specificity for a GAVE6-encoding nucleic acid can be designed based on the nucleotide sequence of a GAVE6 DNA disclosed herein (e.g., SEQ ID NO: 1). For example, a derivative of a Tetrahymena L-19 IVS RNA can be constructed so that the nucleotide sequence of the active site is complementary to the nucleotide sequence to be cleaved in a GAVE6-encoding mRNA, see, e.g., U.S. Pat. Nos. 4,987,071 and 5,116,742. Alternatively, GAVE6 mRNA can be used to select a catalytic RNA having a specific ribonuclease activity from a pool of RNA molecules, see, e.g., Bartel et al., Science (1993) 261:1411-1418.

Triple Helical Nucleic Acid Molecules and Peptide Nucleic Acids of the of the Present Invention

The invention also encompasses nucleic acid molecules that form triple helical structures. For example, GAVE6 gene expression can be inhibited by targeting nucleotide sequences complementary to the regulatory region of the GAVE6 (e.g., the GAVE6 promoter and/or enhancers) to form triple helical structures that prevent transcription of the GAVE6 gene in target cells, see generally, Helene, Anticancer Drug Des (1991) 6(6):569; Helene Ann NY Acad Sci (1992) 660:27; and Maher, Bioassays (1992) 14(12):807.

In particular embodiments, the nucleic acid molecules of the invention can be modified at the base moiety, sugar moiety or phosphate backbone to improve, e.g., the stability, hybridization or solubility of the molecule. For example, the deoxyribose phosphate backbone of the nucleic acids can be modified to generate peptide nucleic acids (see Hyrup et al., Bioorganic & Medicinal Chemistry (1996) 4:5). As used herein, the terms “peptide nucleic acids” or “PNAs” refer to nucleic acid mimics, e.g., DNA mimics, in that the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) supra; Perry-O'Keefe et al., Proc Natl Acad Sci USA (1996) 93:14670.

PNAs of GAVE6 can be used in therapeutic and diagnostic applications. For example, PNAs can be used as antisense or antigene agents for sequence-specific modulation of gene expression by, e.g., inducing transcription or translation arrest or inhibiting replication. PNAs of GAVE6 also can be used. For example, a PNA can be used in the analysis of single base pair mutations in a gene by, e.g., PNA-directed PCR clamping; as artificial restriction enzymes when used in combination with other enzymes, e.g., S1 nucleases (Hyrup et al. (1996) supra) or as probes or primers for DNA sequence and hybridization (Hyrup et al. (1996) supra; Perry-O'Keefe et al. (1996) supra).

In another embodiment, PNAs of GAVE6 can be modified, e.g., to enhance stability, specificity or cellular uptake, by attaching lipophilic or other helper groups to the PNA, by the formation of PNA-DNA chimeras or by the use of liposomes or other techniques of drug delivery known in the art. The synthesis of PNA-DNA chimeras can be performed as described in Hyrup et al. (1996) supra, Finn et al., Nucleic Acids Res (1996) 24(17):3357-63, Mag et al., Nucleic Acids Res (1989) 17:5973; and Peterser et al., Bioorganic Med Chem Lett (1975) 5:1119.

GAVE6 Protein

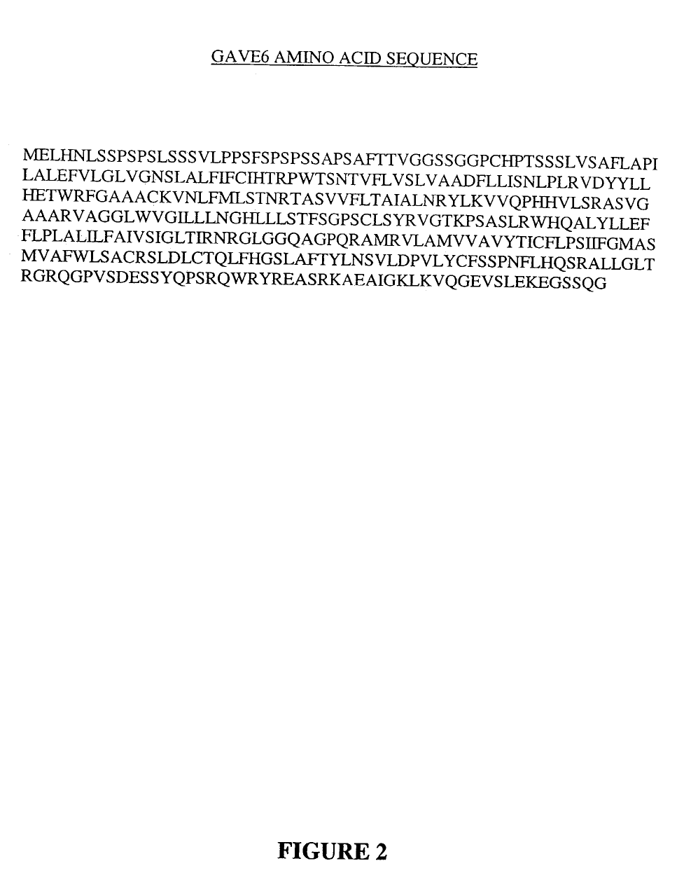

Moreover, the present invention extends to an isolated polypeptide comprising the amino acid sequence of FIG. 2 (SEQ ID NO: 2), a variant thereof, a fragment thereof or an analog or derivative thereof.

An isolated nucleic acid molecule encoding a GAVE6 protein having a sequence that differs from that of SEQ ID NO: 2, e.g. a variant, can be created by introducing one or more nucleotide substitutions, additions or deletions into the nucleotide sequence of SEQ ID NO:l such that one or more amino acid substitutions, additions or deletions are introduced into the encoded protein.

In a particular embodiment, a mutant GAVE6 protein can be assayed for: (1) the ability to form protein:protein interactions with proteins in the GAVE6 signaling pathway; (2) the ability to bind a GAVE6 ligand; or (3) the ability to bind to an intracellular target protein. In yet another embodiment, a mutant GAVE6 can be assayed for the ability to modulate cellular proliferation or cellular differentiation.

Native GAVE6 proteins can be isolated from cells or tissue sources by an appropriate purification scheme using standard protein purification techniques. Alternatively, GAVE6 proteins can readily be produced by recombinant DNA techniques. Yet another alternative encompassed by the present invention is the chemical synthesis of a GAVE6 protein or polypeptide using standard peptide synthesis techniques.

An “isolated” or “purified” protein, or biologically active portion thereof, is substantially free of cellular material or other contaminating proteins from the cell or tissue source from which the GAVE6 protein is derived, or is substantially free of chemical precursors or other chemicals when chemically synthesized. The phrase, “substantially free of cellular material” includes preparations of GAVE6 protein in which the protein is separated from cellular components of the cells from which the protein is isolated or recombinantly produced. Thus, GAVE6 protein that is substantially free of cellular material includes preparations of GAVE6 protein having less than about 30%, 20%, 10% or 5% or less (by dry weight) of non-GAVE6 protein (also referred to herein as a “contaminating protein”). When the GAVE6 protein or biologically active portion thereof is produced recombinantly, it also is preferably substantially free of culture medium, i.e., culture medium represents less than about 20%, 10% or 5% or less of the volume of the protein preparation. When GAVE6 protein is produced by chemical synthesis, it is preferably substantially free of chemical precursors or other chemicals, i.e., it is separated from chemical precursors or other chemicals that are involved in the synthesis of the protein. Accordingly, such preparations of GAVE6 protein have less than about 30%, 20%, 10% or 5% or less (by dry weight) of chemical precursors or non-GAVE6 chemicals.

Biologically active portions or fragments of a GAVE6 protein include peptides comprising amino acid sequences sufficiently identical to or derived from the amino acid sequence of the GAVE6 protein (e.g., the amino acid sequence shown in SEQ ID NO: 2), that include fewer amino acids than the full length GAVE6 protein and exhibit at least one activity of a GAVE6 protein. Typically, biologically active portions comprise a domain or motif with at least one activity of a GAVE6 protein. A biologically active portion of a GAVE6 protein can be a polypeptide that is, for example, 10, 25, 50, 100 or more amino acids in length. Particular biologically active polypeptides include one or more identified GAVE6 structural domains.

Moreover, other biologically active portions, in which other regions of the protein are deleted, can be prepared by recombinant techniques and evaluated for one or more of the functional activities of a native GAVE6 protein.

Other useful GAVE6 proteins are substantially identical to SEQ ID NO: 2 and retain a functional activity of the protein of SEQ ID NO: 2 yet differ in amino acid sequence due to natural allelic variation or mutagenesis. For example, such GAVE6 proteins and polypeptides possess at least one biological activity described herein.

Accordingly, a useful GAVE6 protein is a protein that includes an amino acid sequence at least about 45%, preferably 55%, 65%, 75%, 85%, 95%, 99% or 100%identical to the amino acid sequence of SEQ iD NO: 2 and retains a functional activity of a GAVE6 protein of SEQ ID NO: 2. In a particular embodiment, the GAVE6 protein retains a functional activity of the GAVE6 protein of SEQ ID NO: 2.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions then are compared. When a position in the first sequence is occupied by the same amino acid residue or nucleotide as the corresponding position in the second sequence, then the molecules are considered identical at that position. The percent identity between the two sequences is a function of the number of identical positions shared by the sequences (i.e., percent identity=number of identical positions/total number of positions (e.g., overlapping positions)×100). In one embodiment, the two sequences are the same length.

The determination of percent identity between two sequences can be accomplished using a mathematical algorithm. A particular, non-limiting example of a mathematical algorithm utilized for the comparison of two sequences is the algorithm of Karlin et al., Proc Natl Acad Sci USA (1990) 87:2264, modified as in Karlin et al., Proc Natl Acad Sci USA (1993) 90:5873-5877. Such an algorithm is incorporated into the NBLAST and XBLAST programs of Altschul et al., J Mol Bio (1990) 215:403. BLAST nucleotide searches can be performed with the NBLAST program, for example, score=100, wordlength=12, to obtain nucleotide sequences homologous to a GAVE6 nucleic acid molecule of the present invention. BLAST protein searches can be performed with the XBLAST program, score=50, wordlength=3 to obtain amino acid sequences homologous to a GAVE6 protein molecule of the invention. To obtain gapped alignments for comparison purposes, Gapped BLAST can be utilized as described in Altschul et al., Nucleic Acids Res (1997) 25:3389. Alternatively, PSI-Blast can be used to perform an iterated search that detects distant relationships between molecules. Altschul et al. (1997) supra. When utilizing BLAST, Gapped BLAST and PSI-Blast programs, the default parameters of the respective programs (e.g., XBLAST and NBLAST) can be used, see http://www.ncbi.nlm.nih.gov.

Another particular, non-limiting example of a mathematical algorithm utilized for the comparison of sequences is the algorithm of Myers et al., CABIOS (1988) 4:11-17. Such an algorithm is incorporated into the ALIGN program (version 2.0) that is part of the GCG sequence alignment software package. When utilizing the ALIGN program for comparing amino acid sequences, a PAM120 weight residue table, a gap length penalty of 12 and a gap penalty of 4 may be used.

The percent identity between two sequences can be determined using techniques similar to those described above, with or without allowing gaps. In calculating percent identity, only exact matches are counted.

The present invention further extends to GAVE6 chimeric or fusion proteins. As used herein, a GAVE6 “chimeric protein” or “fusion protein” comprises a GAVE6 polypeptide operably linked to a non-GAVE6 polypeptide. A “GAVE6 polypeptide” refers to a polypeptide having an amino acid sequence corresponding to GAVE6. A “non-GAVE6 polypeptide” refers to a polypeptide having an amino acid sequence corresponding to a protein that is not substantially identical to the GAVE6 protein, e.g., a protein that is different from the GAVE6 protein and is derived from the same or a different organism. Within a GAVE6 fusion protein, the GAVE6 polypeptide can correspond to all or a portion of a GAVE6 protein, preferably at least one biologically active portion of a GAVE6 protein. Within the fusion protein, the term “operably linked” is intended to indicate that the GAVE6 polypeptide and the non-GAVE6 polypeptide are fused in-frame to each other. The non-GAVE6 polypeptide can be fused to the N-terminus or C-terminus of a GAVE6 polypeptide. One useful fusion protein is GST-GAVE6 in which a GAVE6 sequence is fused to the C-terminus of glutathione-S-transferase (GST). Such fusion proteins can facilitate the purification of recombinant GAVE6.

In another embodiment, a fusion protein of the present invention extends to a GAVE6-immunoglobulin fusion protein in which all or part of GAVE6 is fused to sequences derived from a member of the immunoglobulin protein family. The GAVE6-immunoglobulin fusion proteins of the invention can be incorporated into pharmaceutical compositions and administered to a subject to inhibit an interaction between a GAVE6 ligand and a GAVE6 protein on the surface of a cell, thereby to suppress GAVE6-mediated signal transduction in vivo. The GAVE6-immunoglobulin fusion proteins can be used to affect the bioavailability of a GAVE6 cognate ligand. Inhibition of the GAVE6 ligand-GAVE6 interaction may be useful therapeutically, both for treating proliferative and differentiative disorders and for modulating (e.g. promoting or inhibiting) cell survival. Moreover, the GAVE6-immunoglobulin fusion proteins of the invention can be used as immunogens to produce anti-GAVE6 antibodies in a subject, to purify GAVE6 ligands and in screening assays to identify molecules that inhibit the interaction of GAVE6 with a GAVE6 ligand.

In a particular embodiment, a GAVE6 chimeric or fusion protein of the present invention is produced by standard recombinant DNA techniques. For example, DNA fragments coding for the different polypeptide sequences are ligated together in-frame in accordance with conventional techniques, for example, by employing blunt-ended or stagger-ended termini for ligation, restriction enzyme digestion to provide for appropriate termini, filling-in of cohesive ends as appropriate, alkaline phosphatase treatment to avoid undesirable joining and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that subsequently can be annealed and reamplified to generate a chimeric gene sequence (see e.g., Ausubel et al., supra). Moreover, many expression vectors are commercially available that already encode a fusion moiety (e.g., a GST polypeptide). A GAVE6-encoding nucleic acid can be cloned into such an expression vector so that the fusion moiety is linked in-frame to the GAVE6 protein.

Variants

As explained above, the present invention further extends to variants of the GAVE6 protein. For example, mutations may be introduced into the amino acid sequence of SEQ ID NO: 2 using standard techniques, such as site-directed mutagenesis and PCR-mediated mutagenesis. Moreover, conservative amino acid substitutions can be made at one or more predicted non-essential amino acid residues. A “conservative amino acid substitution” is one in which the amino acid residue is replaced with an amino acid residue having a similar side chain. For example, one or more amino acids can be substituted by another amino acid of a similar polarity, which acts as a functional equivalent, resulting in a silent alteration. Substitutes for an amino acid within the amino acid sequence of a polypeptide of the present invention may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to effect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free -OH can be maintained; and

Gln for Asn such that a free NH2 can be maintained.

Moreover, amino acid substitutions may also be introduced to substitute an amino acid with a particularly preferable property. For example, a Cys may be introduced for a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Mutations can also be introduced randomly along all or part of a GAVE6 coding sequence, such as by saturation mutagenesis, and the resultant mutants can be screened for GAVE6 biological activity to identify mutants that retain activity. Following mutagenesis, the encoded protein can be expressed recombinantly and the activity of the protein can be determined.

Variants of the present invention can function as a GAVE6 agonist (mimetic) or as GAVE6 antagonist. Variants of the GAVE6 protein can be generated by mutagenesis, e.g., discrete point mutation or truncation of the GAVE6 protein. An agonist of the GAVE6 protein can retain substantially the same or a subset of the biological activities of the naturally occurring GAVE6 protein. For example, an antagonist of the GAVE6 protein can competitively bind to a downstream or upstream member of a cellular signaling cascade that includes the GAVE6 protein, and thus inhibit one or more of the activities of the naturally occurring form of the GAVE6 protein. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the GAVE6 proteins.

Variants of the GAVE6 protein that function as either GAVE6 agonists (mimetics) or as GAVE6 antagonists can be identified by screening combinatorial libraries of mutants, e.g., truncation mutants, of the GAVE6 protein for GAVE6 agonist or antagonist activity. In one embodiment, a variegated library of GAVE6 variants is generated by combinatorial mutagenesis at the nucleic acid level, and is encoded by a variegated gene library. A variegated library of GAVE6 variants can be produced by, for example, enzymatically ligating a mixture of synthetic oligonucleotides into gene sequences such that a degenerate set of potential GAVE6 sequences is expressed as individual polypeptides or alternatively, as a set of larger fusion proteins (e.g., for phage display) containing the set of GAVE6 sequences therein. There are a variety of methods that can be used to produce libraries of potential GAVE6 variants from a degenerate oligonucleotide sequence. Chemical synthesis of a degenerate gene sequence can be performed in an automated DNA synthesizer and the synthetic gene then ligated into an appropriate expression vector. Use of a degenerate set of genes allows for the provision, in one mixture, of all of the sequences encoding the desired set of potential GAVE6 sequences. Methods for synthesizing degenerate oligonucleotides are known in the art (see, e.g., Narang, Tetrahedron (1983) 39:3; Itakura et al., Ann Rev Biochem (1984) 53:323; Itakura et al., Science (1984) 198:1056; Ike et al., Nucleic Acid Res (1983) 11:477).

In addition, libraries of fragments of the GAVE6 protein coding sequence can be used to generate a variegated population of GAVE6 fragments for screening and subsequent selection of variants of a GAVE6 protein. In one embodiment, a library of coding sequence fragments can be generated by treating a double-stranded PCR fragment of a GAVE6 coding sequence with a nuclease under conditions wherein nicking occurs only about once per molecule, denaturing the double-stranded DNA, renaturing the DNA to form double-stranded DNA that can include sense/antisense pairs from different nicked products, removing single-stranded portions from reformed duplexes by treatment with S1 nuclease and ligating the resulting fragment library into an expression vector. By that method, an expression library can be derived that encodes N-terminal and internal fragments of various sizes of the GAVE6 protein.

Several techniques are known in the art for screening gene products of combinatorial libraries made by point mutations or truncation and for screening cDNA libraries for gene products having a selected property. Such techniques are adaptable for rapid screening of the gene libraries generated by the combinatorial mutagenesis of GAVE6 proteins. The most widely used techniques that are amenable to high through-put analysis for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique that enhances the frequency of functional mutants in the libraries, can be used in combination with the screening assays to identify GAVE6 variants (Arkin et al., Proc Natl Acad Sci USA (1992) 89:7811-7815; Delgrave et al., Protein Engineering (1993) 6(3):327-33 1).

Analogs and Derivatives of GAVE6

Moreover, the present invention also includes derivatives or analogs of GAVE6 produced from a chemical modification. A GAVE6 protein of the present invention may be derivatized by the attachment of one or more chemical moieties to the protein moiety.

Chemical Moieties For Derivatization. The chemical moieties suitable for derivatization may be selected from among water soluble polymers so that the GAVE6 analog or derivative does not precipitate in an aqueous environment, such as a physiological environment. Optionally, the polymer will be pharmaceutically acceptable. One skilled in the art will be able to select the desired polymer based on such considerations as whether the polymer/component conjugate will be used therapeutically, and if so, the desired dosage, circulation time, resistance to proteolysis, and other considerations. For GAVE6, these may be ascertained using the assays provided herein. Examples of water soluble polymers having applications herein include, but are not limited to, polyethylene glycol, copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), dextran, poly(n-vinyl pyrrolidone)polyethylene glycol, propropylene glycol homopolymers, polypropylene oxide/ethylene oxide co-polymers, polyoxyethylated polyols or polyvinyl alcohol. Polyethylene glycol propionaldenhyde may have advantages in manufacturing due to its stability in water.

The polymer may be of any molecular weight, and may be branched or unbranched. For polyethylene glycol, the preferred molecular weight is between about 2 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects if any, on biological activity, the ease in handling, the degree or lack of antigenicity and other known effects of the polyethylene glycol to a therapeutic protein or analog).

The number of polymer molecules so attached to GAVE6 may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to GAVE6 molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

The polyethylene glycol molecules (or other chemical moieties) should be attached to GAVE6 with consideration of effects on functional or antigenic domains of GAVE6. There are a number of attachment methods available to those skilled in the art, e.g., EP 0 401 384 herein incorporated by reference (coupling PEG to G-CSF), see also Malik et al., 1992, Exp. Hematol. 20:1028-1035 (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group include lysine residues and the N-terminal amino acid residues; those having a free carboxyl group include aspartic acid residues, glutamic acid residues and the C-terminal amino acid residue. Sulfhydryl groups may also be used as a reactive group for attaching the polyethylene glycol molecule(s). Preferred for therapeutic purposes is attachment at an amino group, such as attachment at the N-terminus or lysine group.

One may specifically desire N-terminally chemically modified GAVE6. Using polyethylene glycol as an illustration of the present compositions, one may select from a variety of polyethylene glycol molecules (by molecular weight, branching, etc.), the proportion of polyethylene glycol molecules to GAVE6 molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective N-terminal chemical modification may be accomplished by reductive alkylation which exploits differential reactivity of different types of primary amino groups (lysine versus the N-terminal) available for derivatization in GAVE6. Under the appropriate reaction conditions, substantially selective derivatization of GAVE6 at the N-terminus with a carbonyl group containing polymer is achieved. For example, one may selectively N-terminally pegylate GAVE6 by performing the reaction at a pH which allows one to take advantage of the pKₐ differences between the ε-amino groups of the lysine residues and that of the α-amino group of the N-terminal residue of GAVE6. By such selective derivatization, attachment of a water soluble polymer to GAVE6 is controlled: the conjugation with the polymer takes place predominantly at the N-terminus of GAVE6 and no significant modification of other reactive groups, such as the lysine side chain amino groups, occurs. Using reductive alkylation, the water soluble polymer may be of the type described above, and should have a single reactive aldehyde for coupling to GAVE6. Polyethylene glycol proprionaldehyde, containing a single reactive aldehyde, may be used.

Antibodies of GAVE6, Variants Thereof, Fragments Thereof, or Analogs or Derivatives Thereof

An isolated GAVE6 protein or a portion or fragment thereof, can be used as an immunogen to generate antibodies that bind GAVE6 using standard techniques for polyclonal and monoclonal antibody preparation. The term “antibody” as used herein refers to immunoglobulin molecules and immunologically active portions of immunoglobulin molecules, i.e., molecules that contain an antigen-binding site that specifically binds an 15 antigen, such as GAVE6, or a fragment thereof. A molecule that specifically binds to GAVE6 is a molecule that binds GAVE6, but does not substantially bind other molecules in a sample, e.g., a biological sample that naturally contains GAVE6. Examples of immunologically active portions of immunoglobulin molecules include F_{(ab)} and F_{(ab′)2} fragments that can be generated by treating the antibody with an enzyme such as pepsin. The invention provides polyclonal, monoclonal and chimeric antibodies that have GAVE6, a variant thereof, a fragment thereof, or an analog or derivative thereof, as an immunogen. Chimeric antibodies are preferred for use in therapy of human diseases or disorders, since the human or humanized antibodies are much less likely than xenogenic antibodies to induce an immune response, in particular an allergic response, themselves.

The full-length GAVE6 protein can be used or, alternatively, the invention provides antigenic peptide fragments of GAVE6 for use as immunogens. The antigenic peptide of GAVE6 comprises at least 8 (preferably 10, 15, 20, 30 or more) amino acid residues of the amino acid sequence shown in SEQ ID NO: 2 and encompasses an epitope of GAVE6 such that an antibody raised against the peptide forms a specific immune complex with GAVE6.

A GAVE6 immunogen typically is used to prepare antibodies by immunizing a suitable subject, (e.g., rabbit, goat, mouse or other mammal) with the immunogen. An appropriate immunogenic preparation can contain, for example, recombinantly expressed GAVE6 protein or a chemically synthesized GAVE6 polypeptide. The preparation further can include an adjuvant, such as Freund's complete or incomplete adjuvant or similar immunostimulatory agent. Immunization of a suitable subject with an immunogenic GAVE6 preparation induces a polyclonal anti-GAVE6 antibody response.

An antibody of the present invention can be a monoclonal antibody, a polyclonal antibody, or a chimeric antibody. The term “monoclonal antibody” or “monoclonal antibody composition”, as used herein, refers to a population of antibody molecules that contain only one species of an antigen-binding site capable of immunoreacting with a particular epitope of GAVE6. A monoclonal antibody composition thus typically displays a single binding affinity for a particular GAVE6 protein epitope.

Polyclonal anti-GAVE6 antibodies can be prepared as described above by immunizing a suitable subject with a GAVE6 immunogen. The anti-GAVE6 antibody titer in the immunized subject can be monitored over time by standard techniques, such as with an enzyme-linked immunosorbent assay (ELISA) using immobilized GAVE6. If desired, the antibody molecules directed against GAVE6 can be isolated from the mammal (e.g., from the blood) and further purified by well-known techniques, such as protein A chromatography, to obtain the IgG fraction. At an appropriate time after immunization, e.g., when the anti-GAVE6 antibody titers are highest, antibody-producing cells can be obtained from the subject and used to prepare monoclonal antibodies by standard techniques, such as the hybridoma technique originally described by Kohler et al., Nature (1975) 256:495-497, the human B cell hybridoma technique (Kohler et al., Immunol Today (1983) 4:72), the EBV hybridoma technique (Cole et al., Monoclonal Antibodies and Cancer Therapy, (1985), Alan R. Liss, Inc., pp. 77-96) or trioma techniques. The technology for producing hybridomas is well known (see generally Current Protocols in Immunology (1994) Coligan et al., eds., John Wiley & Sons, Inc., New York, N.Y.). Briefly, an immortal cell line (typically a myeloma) is fused to lymphocytes (typically splenocytes) from a mammal immunized with a GAVE6 immunogen as described above and the culture supernatants of the resulting hybridoma cells are screened to identify a hybridoma producing a monoclonal antibody that binds GAVE6.

Any of the many well known protocols used for fusing lymphocytes and immortalized cell lines can be applied for the purpose of generating an anti-GAVE6 monoclonal antibody (see, e.g., Current Protocols in Immunology, supra; Galfre et al., Nature (1977) 266:550-552; Kenneth, in Monoclonal Antibodies: A New Dimension In Biological Analyses, Plenum Publishing Corp., New York, N.Y. (1980); and Lerner, Yale J Biol Med (1981) 54:387-402). Moreover, the ordinarily skilled worker will appreciate that there are many variations of such methods that also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the instant invention with an immortalized mouse cell line, e.g., a myeloma cell line that is sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-×63-Ag8.653 or Sp2/O-Ag14 myeloma lines. The myeloma lines are available from ATCC. Typically, HAT-sensitive mouse myeloma cells are fused to mouse splenocytes using polyethylene glycol (“PEG”). Hybridoma cells resulting from the fusion then are selected using HAT medium that kills unfused and unproductively fused myeloma cells (unfused splenocytes die after several days because they are not transformed). Hybridoma cells producing a monoclonal antibody of the invention are detected by screening the hybridoma culture supernatants for antibodies that bind GAVE6, e.g., using a standard ELISA assay.

Alternative to preparing monoclonal antibody-secreting hybridomas, a monoclonal anti-GAVE6 antibody can be identified and isolated by screening a recombinant combinatorial immunoglobulin library (e.g., an antibody phage display library) with GAVE6 thereby to isolate immunoglobulin library members that bind GAVE6. Kits for generating and screening phage display libraries are commercially available (e.g., the Pharmacia Recombinant Phage Antibody System, Catalog No. 27-9400-01; and the Stratagene “SURFZAP” Phage Display Kit, Catalog No. 240612).

Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology (1991) 9:1370-1372; Hay et al., Hum Antibody Hybridomas (1992) 3:81-85; Huse et al., Science (1989) 246:1275-1281; and Griffiths et al., EMBO J (1993) 25(12):725-734.

Furthermore, recombinant anti-GAVE6 antibodies, such as chimeric and humanized monoclonal antibodies comprising both human and non-human portions, can be made using standard recombinant DNA techniques. Such chimeric and humanized monoclonal antibodies can be produced by recombinant DNA techniques known in the art, for example using methods described in PCT Publication No. WO 87/02671; Europe Patent Application No. 184,187; Europe Patent Application No. 171,496; Europe Patent Application No. 173,494; PCT Publication No. WO 86/01533; U.S. Pat. No. 4,816,567; Europe Patent Application No. 125,023; Better et al., Science (1988) 240:1041-1043; Liu et al., Proc Natl Acad Sci USA (1987) 84:3439-3443; Lin et al., J Immunol (1987) 139:3521-3526; Sun et al., Proc Natl Acad Sci USA (1987) 84:214-218; Nishimura et al., Canc Res (1987) 47:999-1005; Wood et al., Nature (1985) 314:446-449; Shaw et al., J Natl Cancer Inst (1988) 80:1553-1559; Morrison, Science (1985) 229:1202-1207; Oi et al., Bio/Techniques (1986) 4:214; U.S. Pat. No. 5,225,539; Jones et al., Nature (1986) 321:552-525; Verhoeyan et al., Science (1988) 239:1534; and Beidler et al., J Immunol (1988) 141:4053-4060.

Completely human antibodies are particularly desirable for therapeutic treatment of human patients. Such antibodies can be produced using transgenic mice that are incapable of expressing endogenous immunoglobulin heavy and light chains genes, but can express human heavy and light chain genes. The transgenic mice are immunized in the normal fashion with a selected antigen, e.g., all or a portion of GAVE6. Monoclonal antibodies directed against the antigen can be obtained using conventional hybridoma technology. The human immunoglobulin transgenes harbored by the transgenic mice rearrange during B cell differentiation and subsequently undergo class switching and somatic mutation. Thus, using such an epitope, e.g., an antibody that inhibits GAVE6 activity is identified. The heavy chain and the light chain of the non-human antibody are cloned and used to create phage display F_{ab} fragments. For example, the heavy chain gene can be cloned into a plasmid vector so that the heavy chain can be secreted from bacteria. The light chain gene can be cloned into a phage coat protein gene so that the light chain can be expressed on the surface of phage. A repertoire (random collection) of human light chains fused to phage is used to infect the bacteria that express the non-human heavy chain. The resulting progeny phage display hybrid antibodies (human light chain/non-human heavy chain). The selected antigen is used in a panning screen to select phage that bind the selected antigen. Several rounds of selection may be required to identify such phage.

Human light chain genes are isolated from the selected phage that bind the selected antigen. The selected human light chain genes then are used to guide the selection of human heavy chain genes as follows. The selected human light chain genes are inserted into vectors for expression by bacteria. Bacteria expressing the selected human light chains are infected with a repertoire of human heavy chains fused to phage. The resulting progeny phage display human antibodies (human light chain/human heavy chain).

Next, the selected antigen is used in a panning screen to select phage that bind the selected antigen. The selected phage display a completely human antibody that recognizes the same epitope recognized by the original selected, non-human monoclonal antibody. The genes encoding both the heavy and light chains are isolated and can be manipulated further for production of human antibody. The technology is described by Jespers et al. (Bio/Technology (1994) 12:899-903).

An anti-GAVE6 antibody (e.g., monoclonal antibody) can be used to isolate GAVE6 by standard techniques, such as affinity chromatography or immunoprecipitation. An anti-GAVE6 antibody can facilitate the purification of natural GAVE6 from cells and of recombinantly produced GAVE6 expressed in host cells. Moreover, an anti-GAVE6 antibody can be used to detect GAVE6 protein (e.g., in a cellular lysate or cell supernatant) to evaluate the abundance and pattern of expression of the GAVE6 protein. Anti-GAVE6 antibodies can be used diagnostically to monitor protein levels in tissue as part of a clinical testing procedure, for example, to determine the efficacy of a given treatment regimen. Detection can be facilitated by coupling the antibody to a detectable substance, which are described infra.

Detectable Labels

Optionally, isolated nucleic acid molecules of the present invention, polypeptides of the present invention, and antibodies of the present invention, as well as fragments of such moieties, may be detectably labeled. Suitable labels include enzymes, fluorophores (e.g., fluorescene isothiocyanate (FITC), phycoerythrin (PE), Texas red (TR), rhodamine, free or chelated lanthanide series salts, especially Eu³⁺, to name a few fluorophores), chromophores, radioisotopes, chelating agents, dyes, colloidal gold, latex particles, ligands (e.g., biotin), bioluminescent materials, and chemiluminescent agents. When a control marker is employed, the same or different labels may be used for the receptor and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. U.V. light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017). Other direct labels include a radionucleotide, a fluorescent moiety or a luminescent moiety. In addition to these direct labelling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70. 419-439, 1980 and in U.S. Pat. No. 4,857,453.

Other labels for use in the invention include magnetic beads or magnetic resonance imaging labels.

In another embodiment, a phosphorylation site can be created on an isolated polypeptide of the present invention, an antibody of the present invention, or a fragment thereof, for labeling with ³²P, e.g., as described in European Pat. No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthphosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

Recombinant Expression Vectors and Host Cells

Another aspect of the invention pertains to vectors, preferably expression vectors, containing a nucleic acid encoding GAVE6 (or a portion thereof). As explained above, one type of vector is a “plasmid,” which refers to a circular double-stranded DNA loop into which additional DNA segments can be ligated. Another type of vector is a viral vector, wherein additional DNA segments can be ligated into a viral genome. Certain vectors are capable of autonomous replication in a host cell (e.g., bacterial vectors having a bacterial origin of replication and episomal mammalian vectors). Other vectors (e.g., non-episomal mammalian vectors) are integrated into the genome of a host cell on introduction into the host cell and thereby are replicated along with the host genome. Moreover, expression vectors are capable of directing the expression of genes operably linked thereto. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), that serve equivalent functions.

A recombinant expression vector of the invention comprises a nucleic acid molecule of the present invention in a form suitable for expression of the nucleic acid in a host cell. That means a recombinant expression vector of the present invention includes one or more regulatory sequences, selected on the basis of the host cells to be used for expression, that is operably linked to the nucleic acid to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals).

Such regulatory sequences are described, for example, in Goeddel, Gene Expression Technology: Methods in Enzymology Vol. 185, Academic Press, San Diego, Calif. (1990). Regulatory sequences include those that direct constitutive expression of the nucleotide sequence in many types of host cells (e.g., tissue specific regulatory sequences). It will be appreciated by those skilled in the art that the design of the expression vector can depend on such factors as the choice of host cell to be transformed, the level of expression of protein desired etc. The expression vectors of the invention can be introduced into host cells to produce proteins or peptides encoded by nucleic acids as described herein (e.g., GAVE6 proteins, mutant forms of GAVE6, fusion proteins etc.).

A recombinant expression vector of the invention can be designed for expression of GAVE6 in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using phage regulatory elements and proteins, such as, a T7 promoter and/or a T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes and the cognate recognition sequences, include Factor Xa, thrombin and enterokinase. Typical fusion expression vectors include pGEX (Pharmacia Biotech Inc; Smith et al., Gene (1988) 67:31-40), pMAL (New England Biolabs, Beverly, Mass.) and pRITS (Pharmacia, Piscataway, N.J.), that fuse glutathione 5-transferase (GST), maltose E binding protein or protein A, respectively, to the target recombinant protein.

Examples of suitable inducible non-fusion E. coli expression vectors include pTrc (Amann et al., Gene (1988) 69:301-315) and pET 11d (Studier et al., Gene Expression Technology: Methods in Enzymology, Academic Press, San Diego, Calif. (1990) 185:60-89). Target gene expression from the pTrc vector relies on host RNA polymerase transcription from a hybrid trp-lac fusion promoter.

One strategy to maximize recombinant protein expression in E. coli is to express the protein in a host with impaired capacity to cleave proteolytically the recombinant protein (Gottesman, Gene Expression Technology: Methods in Enzymology, Academic Press, San Diego, Calif. (1990) 185:119-128). Another strategy is to alter the nucleic acid sequence of the nucleic acid molecule to be inserted into an expression vector so that the individual codons for each amino acid are those preferentially utilized in E. coli (Wada et al., Nucleic Acids Res (1992) 20:2111-2118). Suchalteration of nucleic acid sequences of the invention can be carried out by standard DNA synthesis techniques.

In another embodiment, the GAVE6 expression vector is a yeast expression vector. Examples of vectors for expression in yeast such as S. cerevisiae include pYepSec1 (Baldari et al., EMBO J (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.).

Alternatively, GAVE6 can be expressed in insect cells using baculovirus expression vectors. Baculovirus vectors available for expression of proteins in cultured insect cells (e.g., Sf9 cells) include the pAc series (Smith et al., Mol Cell Biol (1983) 3:2156-2165) and the pVL series (Lucklow et al., Virology (1989) 170:31-39).

In yet another embodiment, a nucleic acid of the invention is expressed in mammalian cells using a mammalian expression vector. Examples of mammalian expression vectors having applications herein include, but certainly are not limited to pCDM8 (Seed, Nature (1987) 329:840) and pMT2PC (Kaufman et al., EMBO J (1987) 6:187-195). When used in mammalian cells, control functions of the expression vector often are provided by viral regulatory elements. For example, commonly used promoters are derived from polyoma, adenovirus 2, cytomegalovirus and simian virus 40. For other suitable expression systems for both prokaryotic and eukaryotic cells, see chapters 16 and 17 of Sambrook et al., supra.

In another embodiment, a recombinant mammalian expression vector of the present invention is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., Genes Dev (1987) 1:268-277), lymphoid-specific promoters (Calame et al., Adv Immunol (1988) 43:235-275), in particular, promoters of T cell receptors (Winoto et al., EMBO J (1989) 8:729-733) and immunoglobulins (Banerji et al., Cell (1983) 33:729-740; Queen et al., Cell (1983) 33:741-748), neuron-specific promoters (e.g., the neurofilament promoter; Byrne et al., Proc Natl Acad Sci USA (1989) 86:5473-5477), pancreas-specific promoters (Edlund et al., Science (1985) 230:912-916) and mammary gland-specific promoters (e.g., milk whey promoter; U.S. Pat. No. 4,873,316 and Europe Application No. 264,166). Developmentally-regulated promoters also are encompassed, for example the murine hox promoters (Kessel et al., Science (1990) 249:374-379) and the α-fetoprotein promoter (Campes et al., Genes Dev (1989) 3:537-546).

The invention further provides a recombinant expression vector comprising a DNA molecule of the invention cloned into an expression vector in an antisense orientation. That is, the DNA molecule is operably linked to a regulatory sequence in a manner that allows for expression (by transcription of the DNA molecule) of an RNA molecule that is antisense to GAVE6 mRNA. Regulatory sequences operably linked to a nucleic acid cloned in the antisense orientation can be chosen that direct the continuous expression of the antisense RNA molecule in a variety of cell types. For example, viral promoters and/or enhancers or regulatory sequences can be chosen that direct constitutive, tissue-specific or cell type-specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes, see Weintraub et al. (Reviews-Trends in Genetics, Vol. 1(1)1986).

Another aspect of the present invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but still are included within the scope of the term as used herein.

A host cell can be any prokaryotic or eukaryotic cell. For example, GAVE6 protein can be expressed in bacterial cells such as E. coli, insect cells, yeast or mammalian cells (such as Chinese hamster ovary cells (CHO), 293 cells or COS cells). Other suitable host cells are known to those skilled in the art. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, transduction, DEAE-dextran-mediated transfection, lipofection or electroporation.

For stable transfection of mammalian cells, it is known that, depending on the expression vector and transfection technique used, only a small fraction of cells may integrate the foreign DNA into the genome. To identify and to select the integrants, a gene that encodes a selectable marker (e.g., for resistance to antibiotics) generally is introduced into the host cells along with the gene of interest. Preferred selectable markers include those that confer resistance to drugs, such as G418, hygromycin and methotrexate. Nucleic acid encoding a selectable marker can be introduced into a host cell on the same vector as that encoding GAVE6 or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) GAVE6 protein. Accordingly, the invention further provides methods for producing GAVE6 protein using the host cells of the invention. In one embodiment, the method comprises culturing the host cell of invention (into that a recombinant expression vector encoding GAVE6 has been introduced) in a suitable medium such that GAVE6 protein is produced. In another embodiment, the method further comprises isolating GAVE6 from the medium or the host cell.

In another embodiment, GAVE6 comprises an inducible expression system for the recombinant expression of other proteins subcloned in modified expression vectors. For example, host cells comprising a mutated G protein (e.g., yeast cells, Y2 adrenocortical cells and cyc S49, see U.S. Pat. Nos. 6,168,927 B1, 5,739,029 and 5,482,835; Mitchell et al., Proc Natl Acad Sci USA (1992) 89(19):8933-37 and Katada et al., J Biol Chem (1984) 259(6):3586-95) are transduced with a first expression vector comprising a nucleic acid sequence encoding GAVE6, wherein GAVE6 is functionally expressed in the host cells. Even though the expressed GAVE6 is constitutively active, the mutation does not allow for signal transduction; i.e., no activation of a G-protein directed downstream cascade occurs (e.g., no adenylyl cyclase activation). Subsequently, a second expression vector is used to transduce the GAVE6-comprising host cells. The second vector comprises a structural gene that complements the G protein mutation of the host cell (i.e., functional mammalian or yeast Gₛ, Gᵢ, Gₒ, or G_{q}, e.g., see PCT Publication No. WO 97/48820; U.S. Pat. Nos. 6,168,927 B1, 5,739,029 and 5,482,835) in addition to the gene of interest to be expressed by the inducible system. The complementary structural gene of the second vector is inducible; i.e., under the control of an exogenously added component (e.g., tetracycline, IPTG, small molecules etc., see Sambrook et al. supra) that activates a promoter which is operably linked to the complementary structural gene. On addition of the inducer, the protein encoded by the complementary structural gene is functionally expressed such that the constitutively active GAVE6 now will form a complex that leads to appropriate downstream pathway activation (e.g., second messenger formation). The gene of interest comprising the second vector possesses an operably linked promoter that is activated by the appropriate second messenger (e.g., CREB, API elements). Thus, as second messenger accumulates, the promoter upstream from the gene of interest is activated to express the product of said gene. When the inducer is absent, expression of the gene of interest is switched off.

In a particular embodiment, the host cells for the inducible expression system include, but are not limited to, S49 (cyc⁻) cells. While cell lines are contemplated that comprise G-protein mutations, suitable mutants may be artificially produced/constructed (see U.S. Pat. Nos. 6,168,927 B1, 5,739,029 and 5,482,835 for yeast cells).

In a related aspect, the cells are transfected with a vector operably linked to a cDNA comprising a sequence encoding a protein as set forth in SEQ ID NO: 2. The first and second vectors comprising said system are contemplated to include, but are not limited to, pCDM8 (Seed, Nature (1987) 329:840) and pMT2PC (Kaufman et al., EMBO J (1987) 6:187-195), pYepSec1 (Baldari et al., EMBO J (1987) 6:229-234), pMFa (Kurjan et al., Cell (1982) 30:933-943), pJRY88 (Schultz et al., Gene (1987) 54:113-123), pYES2 (Invitrogen Corporation, San Diego, Calif.) and pPicZ (Invitrogen Corp, San Diego, Calif.). In a related aspect, the host cells may be transfected by such suitable means, wherein transfection results in the expression of a functional GAVE6 protein (e.g., Sambrook et al., supra, and Kriegler, Gene Transfer and Expression: A Laboratory Manual, Stockton Press, New York, N.Y., 1990). Such “functional proteins” include, but are not limited to, proteins that once expressed, form complexes with G-proteins, where the G-proteins regulate second messenger formation. Other methods for transfecting host cells that have applications herein include, but certainly are not limited to transfection, electroporation, microinjection, transduction, cell fusion, DEAE dextran, calcium phosphate precipitation, lipofection (lysosome fusion), use of a gene gun, or a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

A large variety of promoters have applications in the present invention. Indeed, expression of a polypeptide of the present invention may be controlled by any promoter/enhancer element known in the art, but these regulatory elements must be functional in the host selected for expression. Promoters which may be used to control GAVE6 expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25); see also “Useful proteins from recombinant bacteria” in Scientific American, 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al., 1984, Cell 38:639-646; Omitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myclin basic protein gene control region which is active in oligodendrocyte cells in the brain (Readhead et al., 1987, Cell 48:703-712), myosin light chain-2 gene control region which is active in skeletal muscle (Sani, 1985, Nature 314:283-286), and gonadotropic releasing hormone gene control region which is active in the hypothalamus (Mason et al., 1986, Science 234:1372-1378).

Expression vectors containing a nucleic acid molecule of the invention can be identified by four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding GAVE6 protein, a variant thereof, or an analog or derivative thereof, is inserted within the “selection marker” gene sequence of the vector, recombinants containing the insert can be identified by the absence of the GAVE6 gene function. In the fourth approach, recombinant expression vectors can be identified by assaying for the activity, biochemical, or immunological characteristics of the gene product expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMa1-C2, pET, pGEX (Smith et al., 1988, Gene 67:31-40), pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible metallothionein Iia gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SamI cloning site, TK- and β-gal selection), pMJ601 (SalI, SamI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamHI, and Hpa cloning site, TK or XPRT selection).

Yeast expression systems can also be used according to the invention to express GAVE6 protein, a variant thereof, or an analog or derivative thereof. For example, the non-fusion pYES2 vector (XbaI, SphI, ShoI, NotI, GstXI, EcoRI, BstXI, BamH1, SacI, KpnI, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors that can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

In addition, a host cell strain may be chosen which modulates the expression of the inserted sequences, or modifies and processes the gene product in the specific fashion desired. Different host cells have characteristic and specific mechanisms for the translational and post-translational processing and modification (e.g., glycosylation, cleavage [e.g., of signal sequence]) of proteins. Appropriate cell lines or host systems can be chosen to ensure the desired modification and processing of the foreign protein expressed. For example, expression in a bacterial system can be used to produce an nonglycosylated core protein product.

Transgenic Animals

A host cell of the present invention also can be used to produce nonhuman transgenic animals. For example, in one embodiment, a host cell of the invention is a fertilized oocyte or an embryonic stem cell into which GAVE6-coding sequences have been introduced. Such host cells then can be used to create non-human transgenic animals into which exogenous GAVE6 sequences have been introduced into the genome, or homologous recombinant animals in which endogenous GAVE6 sequences have been altered. Such animals are useful for studying the function and/or activity of GAVE6 and for identifying and/or evaluating modulators of GAVE6 activity. As used herein, a “transgenic animal” is a non-human animal, preferably a mammal, more preferably a rodent such as a rat or mouse, in that one or more of the cells of the animal includes a transgene. Other examples of transgenic animals include non-human primates, sheep, dogs, cows, goats, chickens, amphibians etc.

As used herein, the term “transgene” refers to exogenous DNA that is integrated into the genome of a cell from which a transgenic animal develops and that remains in the genome of the mature animal. The transgene directs the expression of an encoded gene product in one or more cell types or tissues of the transgenic animal. As used herein, a “homologous recombinant animal” is a non-human animal, preferably a mammal, more preferably a mouse, in which an endogenous GAVE6 gene has been altered by homologous recombination. That is accomplished between the endogenous gene and an exogenous DNA molecule introduced into a cell of the animal, e.g., an embryonic cell of the animal, prior to development of the animal.

A transgenic animal of the invention can be created by introducing a GAVE6-encoding nucleic acid molecule into the male pronuclei of a fertilized oocyte using one of the transfection methods described above. The oocyte is then allowed to develop in a pseudopregnant female foster animal. The GAVE6 cDNA sequence e.g., that of (SEQ ID NO: 1), for example, can be introduced as a transgene into the genome of a non-human animal. Alternatively, a nonhuman homologue of the human GAVE6 gene, such as a mouse GAVE6 gene, can be isolated based on hybridization to the human GAVE6 cDNA, and used as a transgene. Intronic sequences and polyadenylation signals also can be included in the transgene to increase the efficiency of expression of the transgene. A tissue-specific regulatory sequence(s) can be operably linked to the GAVE6 transgene to direct expression of GAVE6 protein in particular cells. Methods for generating transgenic animals via embryo manipulation and microinjection, particularly animals such as mice, are conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals with a transgene in the genome and/or expression of GAVE6 mRNA in tissues or cells of the animals. A transgenic founder animal then can be used to breed additional animals carrying the transgene. Moreover, transgenic animals carrying a transgene encoding GAVE6 can be bred further to other transgenic animals carrying other transgenes.

To create a homologous recombinant animal, a vector is prepared that contains at least a portion of a GAVE6 gene (e.g., a human or a non-human homolog of the GAVE6 gene, e.g., a murine GAVE6 gene) into which a deletion, addition or substitution has been introduced thereby to alter, e.g., functionally disrupt, the GAVE6 gene. In a particular embodiment, the vector is designed such that, on homologous recombination, the endogenous GAVE6 gene is disrupted functionally (i.e., no longer encodes a functional protein; also referred to as a “knock out” vector).

Alternatively, the vector can be designed such that, on homologous recombination, the endogenous GAVE6 gene is mutated or otherwise altered but still encodes functional protein (e.g., the upstream regulatory region can be altered thereby to alter the expression of the endogenous GAVE6 protein).

In the homologous recombination vector, the altered portion of the GAVE6 gene is flanked at the 5′ and 3′ ends by an additional nucleic acid sequence of the GAVE6 gene to allow for homologous recombination to occur between the exogenous GAVE6 gene carried by the vector and an endogenous GAVE6 gene in an embryonic stem cell. The additional flanking GAVE6 nucleic acid sequence is of sufficient length for successful homologous recombination with the endogenous gene. Typically, several kilobases of flanking DNA (both at the 5′ and 3′ ends) are included in the vector (see, e.g., Thomas et al., Cell (1987) 51:503 for a description of homologous recombination vectors).

The vector is introduced into an embryonic stem cell line (e.g., by electroporation) and cells in which the introduced GAVE6 gene has homologously recombined with the endogenous GAVE6 gene are selected (see, e.g., Li et al., Cell (1992) 69:915). The selected cells then are injected into a blastocyst of an animal (e.g., a mouse) to form aggregation chimeras (see, e.g., Bradley in Teratocarcinomas and Embryonic Stem Cells: A Practical Approach, Robertson, ed., IRL, Oxford, (1987) pp. 113-152). A chimeric embryo then can be implanted into a suitable pseudopregnant female foster animal and the embryo brought to term. Progeny harboring the homologously recombined DNA in the germ cells can be used to breed animals in that all cells of the animal contain the homologously recombined DNA by germline transmission of the transgene.

Methods for constructing homologous recombination vectors and homologous recombinant animals are described further in Bradley, Current Opinion in Bio/Technology (1991) 2:823-829 and in PCT Publication Nos. WO 90/11354, WO 91/01140, WO 92/0968 and WO 93/04169.

In another embodiment, transgenic non-human animals can be produced that contain selected systems to allow for regulated expression of the transgene. One example of such a system is the cre/loxP recombinase system of bacteriophage P1. For a description of the cre/loxP recombinase system, see, e.g., Lakso et al., Proc Natl Acad Sci USA (1992) 89:6232-6236.

Another example of a recombinase system is the FLP recombinase system of S. cerevisiae (O'Gorrnan et al., Science (1991) 251:1351-1355). If a cre/loxP recombinase system is used to regulate expression of the transgene, animals containing transgenes encoding both the cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein also can be produced according to the methods described in Wilmut et al., Nature (1997) 385:810-813 and PCT Publication Nos. WO 97/07668 and WO 97/07669. In brief, a cell, e.g., a somatic cell, from the transgenic animal can be isolated and induced to exit the growth cycle and enter G₀ phase. The quiescent cell then can be fused, e.g., through the use of electrical pulses, to an enucleated oocyte from an animal of the same species from which the quiescent cell is isolated. The reconstructed oocyte then is cultured such that it develops to morula or blastocyte, and then is transferred to a pseudopregnant female foster animal. The offspring borne of the female foster animal-will be a clone of the animal from that the cell, e.g., the somatic cell, is isolated.

Pharmaceutical Compositions

The GAVE6 nucleic acid molecules, GAVE6 proteins and anti-GAVE6 antibodies (also referred to herein as “active compounds”) of the present invention can be incorporated into pharmaceutical compositions suitable for administration. Such compositions typically comprise the nucleic acid molecule, protein or antibody, and a pharmaceutically acceptable carrier. As used herein, the language “pharmaceutically acceptable carrier” is intended to include any and all solvents, dispersion media, coatings, antibacterial and antifungal agents, isotonic and absorption delaying agents and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds also can be incorporated into the compositions.

A pharmaceutical composition of the present invention is formulated to be compatible with the intended route of administration. Examples of routes of administration include parenteral, e.g., intravenous, intradermal, subcutaneous, oral (e.g., inhalation), transdermal (topical), transmucosal and rectal administration. Solutions or suspensions used for parenteral, intradermal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. pH can be adjusted with acids or bases, such as HCl or NaOH. The parenteral preparation can be enclosed in ampoules, disposable syringes or multiple dose vials made of glass or plastic.

Pharmaceutical compositions suitable for injectable use include sterile aqueous solutions (water miscible) or dispersions and sterile powders for the extemporaneous preparation of sterile injectable solutions or dispersions. For intravenous administration, suitable carriers include physiological saline, bacteriostatic water, “CREMOPHOR EL” (BASF; Parsippany, N.J.) or phosphate buffered saline (PBS). In all cases, the composition must be sterile and should be fluid to the extent that easy syringability exists. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyetheylene glycol and the like) and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols such as mannitol, sorbitol or sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a GAVE6 protein, variant thereof, or analog or derivative thereof; or an anti-GAVE6 antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze drying to yield a powder of the active ingredient plus any additional desired ingredient from a previously sterile-filtered solution thereof.

Oral compositions generally include an inert diluent or an edible carrier. The compositions can be enclosed in gelatin capsules or compressed into tablets. For the purpose of oral therapeutic administration, the active compound can be incorporated with excipients and used in the form of tablets, troches or capsules. Oral compositions also can be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients or compounds of a similar nature: a binder such as microcrystalline cellulose, gum tragacanth or gelatin; an excipient such as starch or lactose, a disintegrating agent such as alginic acid, Primogel or corn starch; a lubricant such as magnesium stearate or Sterotes; a glidant such as colloidal silicon dioxide; a sweetening agent such as sucrose or saccharin; or a flavoring agent such as peppermint, methyl salicylate or orange flavoring. For administration by inhalation, the compounds are delivered in the form of an aerosol spray from a pressurized container or dispenser that contains a suitable propellant, e.g., a gas such as carbon dioxide or a nebulizer.

Systemic administration also can be by transmucosal or transdermal means. For transmucosal or transdermal administration, penetrants appropriate to the barrier to be permeated are used in the formulation. Such penetrants generally are known in the art and include, for example, for transmucosal administration, detergents, bile salts and fusidic acid derivatives. Transmucosal administration can be accomplished through the use of nasal sprays or suppositories. For transdermal administration, the active compounds are formulated into ointments, salves, gels or creams as generally known in the art.

The compounds also can be prepared in the form of suppositories (e.g., with conventional suppository bases such as cocoa butter and other glycerides) or retention enemas for rectal delivery.

In a particular embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid.

Methods for preparation of such formulations will be apparent to those skilled in the art. The materials also can be obtained commercially from Alza Corporation and Nova Pharmaceuticals, Inc. Liposomal suspensions (including liposomes targeted to infected cells with monoclonal antibodies) also can be used as pharmaceutically acceptable carriers. Those can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. Depending on the type and severity of the disease, about 1 μg/kg to 15 mg/kg (e.g., 0.1 to 20 mg/kg) of compound is an initial candidate dosage for administration to the patient, whether, for example, by one or more separate administrations or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of the therapy is monitored easily by conventional techniques and assays. An exemplary dosing regimen is disclosed in WO 94/04188. The specification for the dosage unit forms of the invention are dictated by and directly dependent on the unique characteristics of the active compound and the particular therapeutic effect to be achieved and the limitations inherent in the art of compounding such an active compound for the treatment of individuals.

Furthermore, a nucleic acid molecule of the present invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al., Proc Natl Acad Sci USA (1994) 91:3054-3057). The pharmaceutical preparation of the gene therapy vector can include the gene therapy vector in an acceptable diluent or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells that produce the gene delivery system.

The pharmaceutical compositions can be included in a container, pack or dispenser together with instructions for administration.

Uses and Methods of the Invention

The nucleic acid molecules, proteins, protein homologues, antibodies of the present invention, and fragments of such moieties, may be used in one or more of the following methods: a) screening assays; b) detection assays (e.g., chromosomal mapping, tissue typing, forensic biology); c) predictive medicine (e.g., diagnostic assays, prognostic assays, monitoring clinical trials and pharmacogenomics); and d) methods of treatment (e.g., therapeutic and prophylactic). A GAVE6 protein interacts with other cellular proteins, and thus can be used for (i) regulation of cellular proliferation; (ii) regulation of cellular differentiation; and (iii) regulation of cell survival. The isolated nucleic acid molecules of the invention can be used to express GAVE6 protein (e.g., via a recombinant expression vector in a host cell in gene therapy applications), to detect GAVE6 mRNA (e.g., in a biological sample) or to detect a genetic lesion in a GAVE6 gene and to modulate GAVE6 activity. In addition, a GAVE6 protein can be used to screen drugs or compounds that modulate GAVE6 activity or expression, as well as to treat disorders characterized by insufficient or excessive production of GAVE6 protein. Screening for the production of GAVE6 protein forms that have decreased or aberrant activity compared to GAVE6 wild type protein can also be performed with the present invention. In addition, an anti-GAVE6 antibody of the invention can be used to detect and to isolate GAVE6 proteins and to modulate GAVE6 activity. The invention further pertains to novel agents identified by the above-described screening assays and uses thereof for treatments as described herein.

Screening Assays

Activation of a G protein receptor in the presence of endogenous ligand allows for G protein receptor complex formation, thereupon leading to the binding of GTP to the G protein. The GTPase domain of the G protein slowly hydrolyzes the GTP to GDP resulting, under normal conditions, in receptor deactivation. However, constitutively activated receptors continue to hydrolyze GDP to GTP.

A non-hydrolyzable substrate of G protein, [³⁵S]GTPγS, can be used to monitor enhanced binding to membranes which express constitutively activated receptors. Traynor and Nahorski reported that [³⁵S]GTPγS can be used to monitor G protein coupling to membranes in the absence and presence of ligand (Traynor et al., Mol Pharmacol (1995) 47(4):848-54). A preferred use of such an assay system is for initial screening of candidate compounds, since the system is generically applicable to all G protein-coupled receptors without regard to the particular G protein that binds to the receptor.

Gₛ₂₀ stimulates the enzyme adenylyl cyclase, while Gᵢ and G₀ inhibit that enzyme. As is well known the art, adenylyl cyclase catalyzes the conversion of ATP to cAMP; thus, constitutively activated GPCRs that couple the Gₛ protein are associated with increased cellular levels of cAMP. Alternatively, constitutively activated GCPRs that might couple the Gᵢ (or Gₒ) protein are associated with decreased cellular levels of cAMP. See “Indirect Mechanism of Synaptic Transmission”, Chpt. 8, from Neuron to Brain (3^{rd} Ed.), Nichols et al. eds., Sinauer Associates, Inc., 1992. Thus, assays that detect cAMP can be used to determine if a candidate compound is an inverse agonist to the receptor. A variety of approaches known in the art for measuring cAMP can be utilized. In one embodiment, anti-cAMP antibodies are used in an ELISA-based format. In another embodiment, a whole cell second messenger reporter system assay is contemplated (see PCT Publication No. WO 00/22131).

In a related aspect, cyclic AMP drives gene expression by promoting the binding of a cAMP-responsive DNA binding protein or transcription factor (CREB) which then binds to the promoter at specific sites called cAMP response elements, and drives the expression of the gene. Thus, reporter systems can be constructed which have a promoter containing multiple cAMP response elements before the reporter gene, e.g., β-galactosidase or luciferase. Further, as a constitutively activated Gₛ-linked receptor causes the accumulation of cAMP, that then activates the gene and expression of the reporter protein. The reporter protein, such as β-galactosidase or luciferase, then can be detected using standard biochemical assays (PCT Publication No. WO 00/22131).

Other G proteins, such as Gₒ and G_{q}, are associated with activation of the enzyme, phospholipase C, which in turn hydrolyzes the phospholipid, PIP2, releasing two intracellular messengers: diacylglycerol (DAG) and inositol 1,4,5-triphosphate (IP3). Increased accumulation of IP3 is associated with activation of G_{q}-associated receptors and Gₒ-associated receptors (PCT Publication No. WO 00/22131). Assays that detect IP3 accumulation can be used to determine if a candidate compound is an inverse agonist to a G_{q}-associated receptor or a Gₒ-associated receptor. G_{q}-associated receptors also can be examined using an AP1 reporter assays that measures whether G_{q}-dependent phospholipase C causes activation of genes containing AP1 elements. Thus, activated G_{q}-associated receptors will demonstrate an increase in the expression of such genes, whereby inverse agonists will demonstrate a decrease in such expression.

Also provided herein is a method (also referred to herein as a “screening assay”) for identifying modulators, i.e., candidate or test compounds or agents (e.g., peptides, peptidomimetics, small molecules or other drugs) that bind to GAVE6 proteins or have a stimulatory or inhibitory effect on, for example, GAVE6 expression or GAVE6 activity.

In one embodiment, the invention provides assays for screening candidate or test compounds that bind to or modulate the activity of the membrane-bound form of a GAVE6 protein, polypeptide or biologically active portion thereof. The test compounds of the instant invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des (1997) 12:145).

Examples of methods for the synthesis of molecular libraries can be found in the art, for example in: DeWitt et al., Proc Natl Acad Sci USA (1993) 90:6909; Erb et al., Proc Natl Acad Sci USA (1994) 91:11422; Zuckermann et al., J Med Chem (1994) 37:2678; Cho et al., Science (1993) 261:1303; Carrell et al., Angew Chem Int Ed Engl (1994) 33:2059; Carell et al., Angew Chem Int Ed Engl (1994) 33:2061; and Gallop et al., J Med Chem (1994) 37:1233.

Libraries of compounds may be presented in solution (e.g., Houghten Bio/Techniques (1992) 13:412-421) or on beads (Lam, Nature (1991) 354:82-84), chips (Fodor, Nature (1993) 364:555-556), bacteria (U.S. Pat. No. 5,223,409), spores (U.S. Pat. Nos. 5,571,698; 5,403,484; and 5,223,409), plasmids (Cull et al., Proc Natl Acad Sci USA (1992) 89:1865-1869) or phage (Scott et al., Science (1990) 249:386-390; Devlin, Science (1990) 249:404-406; Cwirla et al., Proc Natl Acad Sci USA (1990) 87:6378-6382; and Felici, J Mol Biol (1991) 222:301-310).

In a particular embodiment of the present invention, an assay is a cell-based assay in which a cell that expresses a membrane-bound form of GAVE6 protein, or a biologically active portion thereof, on the cell surface is contacted with a test compound and the ability of the test compound to bind to a GAVE6 protein is determined. The cell, for example, can be a yeast cell or a cell of mammalian origin. Determining the ability of the test compound to bind to the GAVE6 protein can be accomplished, for example, by coupling the test compound with a radioisotope or enzymatic label so that binding of the test compound to the GAVE6 protein or biologically active portion thereof can be determined by detecting the labeled compound in a complex. For example, test compounds can be labeled with ¹²⁵I, ³⁵S, ¹⁴C or ³H, either directly or indirectly and the radioisotope detected by direct counting of radioemmission or by scintillation counting. Alternatively, test compounds can be labeled enzymatically with, for example, horseradish peroxidase, alkaline phosphatase or luciferase and the enzymatic label detected by determination of conversion of an appropriate substrate to product. In a particular embodiment, the assay comprises contacting a cell that expresses a membrane-bound form of GAVE6 protein or a biologically active portion thereof, on the cell surface with a known compound that binds GAVE6 to form an assay mixture, contacting the assay mixture with a test compound and determining the ability of the test compound to interact with a GAVE6 protein, wherein determining the ability of the test compound to interact with a GAVE6 protein comprises determining the ability of the test compound to bind preferentially to GAVE6 or a biologically active portion thereof as compared to the known compound.

In another embodiment, an assay is a cell-based assay comprising contacting a cell expressing a membrane-bound form of GAVE6 protein or a biologically active portion thereof, on the cell surface with a test compound and determining the ability of the test compound to modulate (e.g., stimulate or inhibit) the activity of the GAVE6 protein or biologically active portion thereof. Determining the ability of the test compound to modulate the activity of GAVE6 or a biologically active portion thereof can be accomplished, for example, by determining the ability of the GAVE6 protein to bind to or to interact with a GAVE6 target molecule. As used herein, a “target molecule” is a molecule with which a GAVE6 protein binds or interacts in nature, for example, a molecule on the surface of a cell that expresses a GAVE6 protein, a molecule on the surface of a second cell, a molecule in the extracellular milieu, a molecule associated with the internal surface of a cell membrane or a cytoplasmic molecule. A GAVE6 target molecule can be a non-GAVE6 molecule or a GAVE6 protein or polypeptide of the instant invention. In one embodiment, a GAVE6 target molecule is a component of a signal transduction pathway that facilitates transduction of an extracellular signal (e.g., a signal generated by binding of a compound to a membrane-bound GAVE6 molecule) through the cell membrane and into the cell. The target, for example, can be a second intercellular protein that has catalytic activity or a protein that facilitates the association of downstream signaling molecules with GAVE6.

Determining the ability of the GAVE6 protein to bind to or to interact with a GAVE6 target molecule can be accomplished by one of the methods described above for determining direct binding. In a particular embodiment, determining the ability of the GAVE6 protein to bind to or to interact with a GAVE6 target molecule can be accomplished by determining the activity of the target molecule. For example, the activity of the target molecule can be determined by detecting induction of a cellular second messenger of the target (e.g., intracellular Ca²⁺, diacylglycerol, IP3 etc.), detecting catalytic/enzymatic activity of the target on an appropriate substrate, detecting the induction of a reporter gene (e.g., a GAVE6-responsive regulatory element operably linked to a nucleic acid encoding a detectable marker, e.g. luciferase) or detecting a cellular response, e.g., cellular differentiation or cell proliferation.

The present invention further extends to a cell-free assay comprising contacting a GAVE6 protein, or biologically active portion thereof, with a test compound, and determining the ability of the test compound to bind to the GAVE6 protein or biologically active portion thereof. Binding of the test compound to the GAVE6 protein can be determined either directly or indirectly as described above. In a preferred embodiment, the assay includes contacting the GAVE6 protein or biologically active portion thereof with a known compound that binds GAVE6 to form an assay mixture, contacting the assay mixture with a test compound, and determining the ability of the test compound to interact with a GAVE6 protein, wherein determining the ability of the test compound to interact with a GAVE6 protein comprises determining the ability of the test compound to preferentially bind to GAVE6 or biologically active portion thereof as compared to the known compound.

Another cell-free assay of the present invention involves contacting GAVE6 protein or biologically active portion thereof, with a test compound and determining the ability of the test compound to modulate (e.g., stimulate or inhibit) the activity of the GAVE6 protein or biologically active portion thereof. Determining the ability of the test compound to modulate the activity of GAVE6 can be accomplished, for example, by determining the ability of the GAVE6 protein to bind to a GAVE6 target molecule by one of the methods described above for determining direct binding. In an alternative embodiment, determining the ability of the test compound to modulate the activity of GAVE6 can be accomplished by determining the ability of the GAVE6 protein to further modulate a GAVE6 target molecule. For example, the catalytic/enzymatic activity of the target molecule on an appropriate substrate can be determined as described previously.

Still another cell-free assay of the present invention comprises contacting the GAVE6 protein or biologically active portion thereof, with a known compound that binds GAVE6 to form an assay mixture, contacting the assay mixture with a test compound and determining the ability of the test compound to interact with a GAVE6 protein. The step for determining the ability of the test compound to interact with a GAVE6 protein comprises determining the ability of the GAVE6 protein preferentially to bind to or to modulate the activity of a GAVE6 target molecule.

Receptors can be activated by non-ligand molecules that necessarily do not inhibit ligand binding but cause structural changes in the receptor to enable G protein binding or, perhaps receptor aggregation, dimerization or clustering that can cause activation. For example, antibodies can be raised to the various portions of GAVE6 that are exposed at the cell surface. Those antibodies activate a cell via the G protein cascade as determined by standard assays, such as monitoring cAMP levels or intracellular Ca⁺² levels. Because molecular mapping, and particularly epitope mapping, is involved, monoclonal antibodies may be preferred. The monoclonal antibodies can be raised both to intact receptor expressed at the cell surface and peptides known to form at the cell surface. The method of Geysen et al., U.S. Pat. No. 5,998,577, can be practiced to obtain a plurality of relevant peptides. Antibodies found to activate GAVE6 may be modified to minimize activities extraneous to GAVE6 activation, such as complement fixation. Thus, the antibody molecules can be truncated or mutated to minimize or to remove activities outside of GAVE6 activation. For example, for certain antibodies, only the antigen-binding portion is needed. Thus, the F_{c} portion of the antibody can be removed.

Cells expressing GAVE6 are exposed to antibody to activate GAVE6. Activated cells then are exposed to various molecules in order to identify which molecules modulate receptor activity, and result in higher activation levels or lower activation levels. Molecules that achieve those goals then can be tested on cells expressing GAVE6 without antibody to observe the effect on non-activated cells. The target molecules then can be tested and modified as candidate drugs for the treatment of disorders associated with altered GAVE6 metabolism using known techniques.

The cell-free assays of the instant invention are amenable to use of both the soluble form and the membrane-bound form of GAVE6. In the case of cell-free assays comprising the membrane-bound form of GAVE6, it may be desirable to utilize a solubilizing agent such that the membrane-bound form of GAVE6 is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON X-100, TRITON X-114, THESIT, isotridecylpoly(ethylene glycol ether)ₙ, 3-[(3-cholamidopropyl)dimethylammino]-1-propane sulfonate (CHAPS), 3-[(3-cholamidopropyl)dimethylammino]-2-hydroxy-1-propane sulfonate (CHAPSO) or N-dodecyl=N,N-dimethyl-3-ammonio-1-propane sulfonate.

In more than one embodiment of the above assay methods of the instant invention, it may be desirable to immobilize either GAVE6 or a target molecule thereof to facilitate separation of complexed from uncomplexed forms of one or both of the proteins, as well as to accommodate automation of the assay. Binding of a test compound to GAVE6 or interaction of GAVE6 with a target molecule in the presence and absence of a candidate compound, can be accomplished in any vessel suitable for containing the reactants. Examples of such vessels include microtitre plates, test tubes and micro-centrifuge tubes. In one embodiment, a fusion protein can be provided that adds a domain that allows one or both of the proteins to be bound to a matrix. For example, glutathione-S-transferase/GAVE6 fusion proteins or glutathione-S-transferase/target fusion proteins can be adsorbed onto glutathione SEPHAROSE beads (Sigma Chemical, St. Louis, Mo.). Alternatively, glutathione-derivatized microtitre plates are then combined with the test compound or the test compound. Subsequently, either the non-adsorbed target protein or GAVE6 protein and the mixture are incubated under conditions conducive to complex formation (e.g., at physiological conditions for salt and pH). Following incubation, the beads or microtitre plate wells are washed to remove any unbound components, and the presence of complex formation is measured either directly or indirectly. Alternatively, the complexes can be dissociated from the matrix and the level of GAVE6 binding or activity determined using standard techniques.

Other techniques for immobilizing proteins on matrices can also be used in the screening assays of the invention. For example, either GAVE6 or a target molecule thereof can be immobilized utilizing conjugation of biotin and streptavidin. Biotinylated GAVE6 or target molecules can be prepared from biotin-NHS (N-hydroxy-succinimide) using techniques well known in the art (e.g., biotinylation kit, Pierce Chemicals, Rockford, Ill.) and immobilized in the wells of streptavidin-coated 96-well plates (Pierce Chemicals). Alternatively, antibodies that are reactive with GAVE6 or a target molecule, but do not interfere with binding of the GAVE6 protein to the target molecule, can be derivatized to the wells of the plate. Upon incubation, unbound target or GAVE6 can be trapped in the wells by antibody conjugation. Methods for detecting such complexes, in addition to those described above for the GST-immobilized complexes, include immunodetection of complexes using antibodies reactive with GAVE6 or target molecule, as well as enzyme-linked assays that rely on detecting an enzymatic activity associated with the GAVE6 or target molecule.

In another embodiment, modulators of GAVE6 expression are identified in a method wherein a cell is contacted with a candidate compound, and the expression of GAVE6 mRNA or protein in the cell is determined. The level of expression of GAVE6 mRNA or protein in the presence of the candidate compound is compared to the level of expression of GAVE6 mRNA or protein in the absence of the candidate compound. The candidate compound then can be identified as a modulator of GAVE6 expression based on that comparison. For example, when expression of GAVE6 mRNA or protein is greater (statistically significantly greater) in the presence of the candidate compound than in the absence thereof, the candidate compound is identified as a stimulator or agonist of GAVE6 mRNA or protein expression. Alternatively, when expression of GAVE6 mRNA or protein is less (statistically significantly less) in the presence of the candidate compound than in the absence thereof, the candidate compound is identified as an inhibitor or antagonist of GAVE6 mRNA or protein expression. If GAVE6 activity is reduced in the presence of ligand or agonist, or in a constitutive GAVE6, below baseline, the candidate compound is identified as an inverse agonist. The level of GAVE6 mRNA or protein expression in the cells can be determined by methods described herein for detecting GAVE6 mRNA or protein.

In yet another aspect of the invention, the GAVE6 proteins can be used as “bait proteins” in a two-hybrid assay or three-hybrid assay (see, e.g., U.S. Pat. No. 5,283,317; Zervos et al., Cell (1993) 72:223-232; Madura et al., J Biol Chem (1993) 268:12046-12054; Bartel et al., Bio/Techniques (1993) 14:920-924; Iwabuchi et al., Oncogene (1993) 8:1693-1696; and PCT Publication No. WO 94/10300), to identify other proteins that bind to or interact with GAVE6 (“GAVE6-binding proteins” or “GAVE6-bp”), and modulate GAVE6 activity. Such GAVE6-binding proteins are also likely to be involved in the propagation of signals by the GAVE6 proteins such as, for example, upstream or downstream elements of the GAVE6 pathway.

Since the present invention enables the production of large quantities of pure GAVE6, physical characterization of the conformation of areas of likely function can be ascertained for rational drug design. For example, the IC3 region of the molecule and EC domains are regions of particular interest. Once the shape and ionic configuration of a region is discerned, candidate drugs that should interact with those regions can be configured and then tested in intact cells, animals and patients. Methods that would enable deriving such 3-D structure information include X-ray crystallography, NMR spectroscopy, molecular modeling and so on. The 3-D structure also can lead to identification of analogous conformational sites in other known proteins where known drugs that act at site exist. Those drugs, or derivatives thereof, may find use with GAVE6.

The invention further pertains to novel agents identified by the above-described screening assays and uses thereof for treatments as described herein.

Assays of the Present Invention

A. Detection Assays

Portions or fragments of the DNA sequences of the present invention can be used in numerous ways as polynucleotide reagents. For example, the sequences can be used to: (i) map the respective genes on a chromosome and, thus, locate gene regions associated with genetic disease; (ii) identify an individual from a minute biological sample (tissue typing); and (iii) aid in forensic identification of a biological sample. The applications are described in the subsections below.

I . Chromosome Mapping

Once the sequence (or a portion of the sequence) of a gene has been isolated, the sequence can be used to map the location of the GAVE6 gene on a chromosome. Accordingly, GAVE6 nucleic acid molecules described herein or fragments thereof can been used to map the location of GAVE6 in a genome. The mapping of the location of the GAVE6 sequence in a genome, particularly a human genome, is an important first step in correlating the sequences with genes associated with disease.

Briefly, GAVE6 genes can be mapped in a genome by preparing PCR primers (preferably 15-25 bp in length) from the GAVE6 sequences. The primers are used for PCR screening of somatic cell hybrids containing individual human chromosomes. Only those hybrids containing the human gene corresponding to the GAVE6 sequences yield an amplified fragment.

Somatic cell hybrids are prepared by fusing somatic cells from different mammals (e.g., human and mouse cells). As hybrids of human and mouse cells grow and divide, generally human chromosomes are lost in random order, but the mouse chromosomes are retained. By using media in which mouse cells cannot grow (because of lack of a particular enzyme), but in which human cells can grow, the one human chromosome that contains the gene encoding the needed enzyme will be retained. By using various media, panels of hybrid cell lines are established. Each cell line in a panel contains either a single human chromosome or a small number of human chromosomes and a full set of mouse chromosomes, allowing easy mapping of individual genes to specific human chromosomes. (D'Eustachio et al., Science (1983) 220:919-924). Somatic cell hybrids containing only fragments of human chromosomes also can be produced by using human chromosomes with translocations and deletions.

PCR mapping of somatic cell hybrids is a rapid procedure for assigning a particular sequence to a particular chromosome. Three or more sequences can be assigned per day using a single thermocycler.

Other mapping strategies that can similarly be used to map a GAVE6 sequence to a particular chromosome in a genome include in situ hybridization (described in Fan et al., Proc Natl Acad Sci USA (1990) 87:6223-27), pre-screening with labeled flow-sorted chromosomes and pre-selection by hybridization to chromosome-specific cDNA libraries.

Fluorescence in situ hybridization (FISH) of a DNA sequence to a metaphase chromosomal spread can also be used to provide a precise chromosomal location in one step. Chromosome spreads can be made using cells in which division has been blocked in metaphase by a chemical, e.g., colcemid, that disrupts the mitotic spindle. The chromosomes can be treated briefly with trypsin and then stained with Giemsa. A pattern of light and dark bands develops on each chromosome so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases and more preferably, 2,000 bases will suffice to get good results in a reasonable amount of time. For a review of the technique, see Verma et a]. (Human Chromosomes: A Manual of Basic Techniques (Pergamon Press, New York, 1988)). Chromosomal mapping can be inferred in silico, and employing statistical considerations, such as lod scores or mere proximity.

Reagents for chromosome mapping can be used individually to locate a single site on a chromosome. Furthermore, panels of reagents can be used for marking multiple sites and/or multiple chromosomes. Reagents corresponding to flanking regions of the GAVE6 gene actually are preferred for mapping purposes. Coding sequences are more likely to be conserved within gene families, thus increasing the chance of cross hybridization during chromosomal mapping.

Once a sequence has been mapped to a precise chromosomal location, the physical position of the sequence on the chromosome can be correlated with genetic map data. (Such data are found, for example, in McKusick, Mendelian Inheritance in Man, available on-line through Johns Hopkins University, Welch Medical Library). The relationship between genes and disease, mapped to the same chromosomal region, can then be identified through linkage analysis (co-inheritance of physically adjacent genes), described in, e.g., Egeland et al., Nature (1987) 325:783-787.

Moreover, differences in the DNA sequences between individuals affected and unaffected with a disease associated with GAVE6 can be determined. If a mutation is observed in some or all of the affected individuals, but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

A GAVE6 sequence of the present invention also can be used to identify individuals from minute biological samples. The United States military, for example, is considering the use of restriction fragment length polymorphism (RFLP) for identification of personnel. In the technique, genomic DNA of an individual is digested with one or more restriction enzymes and probed on a Southern blot to yield unique bands for identification. The method does not suffer from the current limitations of “Dog Tags” that can be lost, switched or stolen, making positive identification difficult. The sequences of the instant invention are useful as additional DNA markers for RFLP (described in U.S. Pat. No. 5,272,057).

Furthermore, the sequences of the instant invention can be used to provide an alternative technique that determines the actual base-by-base DNA sequence of selected portions of the genome of an individual. Thus, a GAVE6 sequence described herein can be used to prepare two PCR primers from the 5′ and 3′ ends of the sequences. The primers then can be used to amplify the DNA of an individual and subsequently provide a sequence thereof.

Panels of corresponding DNA sequences from individuals, prepared in that manner, can provide unique individual identifications, as each individual will have a unique set of such DNA sequences due to allelic differences. The sequences of the instant invention can be used to obtain such identification sequences from individuals and from tissue. GAVE6 sequence of the invention uniquely represents portions of the human genome. Allelic variation occurs to some degree in the coding regions of the sequences and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1 can provide positive individual identification with a panel of perhaps 10 to 1,000 primers that each yield a noncoding amplified sequence of 100 bases. If predicted coding sequences, such as those in SEQ ID NO: 1 are used, a more appropriate number of primers for positive individual identification would be 500-2,000.

If a panel of reagents from GAVE6 sequences described herein is used to generate a unique identification database for an individual, those same reagents can later be used to identify tissue from that individual. Using the unique identification database, positive identification of the individual, living or dead, can be made from extremely small tissue samples.

3. Use of Partial GAVE6 Sequences in Forensic Biology

DNA-based identification techniques also can be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva or semen found at a crime scene. The amplified sequence then can be compared to a standard, thereby allowing identification of the origin of the biological sample.

The sequences of the instant invention can be used to provide polynucleotide reagents, e.g., PCR primers, targeted to specific loci in the human genome, that can enhance the reliability of DNA-based forensic identifications. For example, a nucleic acid of interest can provide another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions of SEQ ID NO: 1 are particularly appropriate for that use as greater numbers of polymorphisms occur in the noncoding regions, enhancing discrimination to differentiate individuals using that technique. Examples of polynucleotide reagents, include the GAVE6 sequences or portions thereof, e.g., fragments derived from the noncoding regions of SEQ ID NO: 1 having a length of at least 20 or 30 bases.

The GAVE6 sequences described herein further can be used to provide polynucleotide reagents, e.g., labeled or labelable probes that can be used in, for example, an in situ hybridization technique, to identify a specific tissue, e.g., brain tissue. Such polynucleotide reagents can be very useful in cases in which a forensic pathologist is presented with a tissue of unknown origin. Panels of such GAVE6 probes can be used to identify tissue by species and/or by organ type.

In a similar fashion, the reagents, e.g., GAVE6 primers or probes, can be used to screen tissue culture for contamination (i.e., screen for the presence of a mixture of different types of cells in a culture).

B. Predictive Medicine

The instant invention also pertains to the field of predictive medicine in that diagnostic assays, prognostic assays, pharmacogenomics and monitoring clinical trials are used for prognostic (predictive) purposes to treat an individual prophylactically. Accordingly, one aspect of the present invention relates to diagnostic assays for determining GAVE6 protein and/or nucleic acid expression as well as GAVE6 activity in the context of a biological sample (e.g., blood, urine, feces, sputum, serum, cells and tissue). The assay can be used to determine whether an individual is afflicted with a disease or disorder, or is at risk of developing a disorder, associated with aberrant GAVE6 expression or activity.

The invention also provides for prognostic (or predictive) assays for determining whether an individual is at risk of developing a disorder associated with GAVE6 protein, nucleic acid expression, or activity. For example, mutations in a GAVE6 gene can be assayed in a biological sample. Such assays can be used for prognostic or predictive purpose thereby to treat prophylactically an individual prior to the onset of a disorder characterized by or associated with GAVE6 protein, nucleic acid expression or activity.

Another aspect of the invention provides methods for determining GAVE6 protein, nucleic acid expression or GAVE6 activity in an individual thereby to select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of GAVE6 in clinical trials. Those and other agents are described in further detail in the following sections.

1. Diagnostic Assays

An exemplary method for detecting the presence or absence of GAVE6 in a biological sample involves obtaining a biological sample from a test subject and contacting the biological sample with a compound or an agent capable of detecting GAVE6 protein or nucleic acid (e.g., mRNA or genomic DNA) that encodes GAVE6 protein such that the presence of GAVE6 is detected in the biological sample. A preferred agent for detecting GAVE6 mRNA or genomic DNA is a labeled nucleic acid probe capable of hybridizing to GAVE6 mRNA or genomic DNA. The nucleic acid probe can be, for example, a full-length GAVE6 nucleic acid, such as the nucleic acid of SEQ ID NO: 1 or a portion thereof, such as an oligonucleotide of at least 15, 30, 50, 100, 250 or 500 or more nucleotides in length and sufficient to specifically hybridize under stringent conditions to GAVE6 mRNA or genomic DNA. Other suitable probes for use in the diagnostic assays of the invention are described herein.

A particular agent for detecting GAVE6 protein is an antibody capable of binding to GAVE6 protein, preferably an antibody with a detectable label. Antibodies can be polyclonal, chimeric, or more preferably, monoclonal. An intact antibody or a fragment thereof (e.g., F_{ab} or F_{(ab′)2}) can be used. The term “biological sample” is intended to include tissues, cells and biological fluids isolated from a subject, as well as tissues, cells and fluids present within a subject. That is, the detection method of the invention can be used to detect GAVE6 mRNA, protein or genomic DNA in a biological sample in vitro as well as in vivo. For example, in vitro techniques for detection of GAVE6 mRNA include Northern hybridization and in situ hybridization. In vitro techniques for detection of GAVE6 protein include ELISA, Western blot, immunoprecipitation and immunofluorescence. In vitro techniques for detection of GAVE6 genomic DNA include Southern hybridization. Furthermore, in vivo techniques for detection of GAVE6 protein include introducing into a subject a labeled anti-GAVE6 antibody. For example, the antibody can be labeled with a radioactive marker, the presence and location of which in a subject can be detected by standard imaging techniques.

In an embodiment, the biological sample contains protein molecules from the test subject. Alternatively, the biological sample can contain mRNA molecules from the test subject or genomic DNA molecules from the test subject. A particular biological sample having applications herein is a peripheral blood leukocyte sample isolated by conventional means from a subject.

Hence, association with a disease and identification of nucleic acid or protein polymorphism diagnostic for the carrier or the affected can be beneficial in developing prognostic or diagnostic assays. For example, it would be beneficial to have a prognostic or diagnostic assay for rheumatoid arthritis, asthma, Crohn's Disease and so on. GAVE6 expression is elevated in cells associated with activated or inflammatory states. Disorders associated with inflammation include, anaphylactic states, colitis, Crohn's Disease, edematous states, contact hypersensitivity, allergy, other forms of arthritis, meningitis and other conditions wherein the immune system reacts to an insult by vascular dilation, heat, collecting cells, fluids and the like at a site resulting in swelling and the like. Thus, a disorder in GAVE6 metabolism may be diagnostic for rheumatoid arthritis. Moreover, the molecular mechanism of rheumatoid arthritis may be detectable, such as, there may be a diagnostic SNP, RFLP, variability of expression level, variability of function and so on, that can be detectable in a tissue sample, such as a blood sample.

In another embodiment, the methods further involve obtaining a biological sample from a control subject, contacting the control sample with a compound or agent capable of detecting GAVE6 protein, mRNA or genomic DNA, such that the presence and amount of GAVE6 protein, mRNA or genomic DNA is detected in the biological sample, and then comparing the presence and amount of GAVE6 protein, mRNA or genomic DNA in the control sample with the presence and amount of GAVE6 protein, mRNA or genomic DNA in a test sample.

High Throughput Assays of Chemical Libraries

Any of the assays for compounds capable of modulating the activity of GAVE6 are amenable to high throughput screening. High throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high thruput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

Kits

The invention also encompasses kits for detecting the presence of GAVE6 in a biological sample (a test sample). Such kits can be used to determine if a subject is suffering from or is at increased risk of developing a disorder associated with aberrant expression of GAVE6 (e.g., an immunological disorder). For example, the kit can comprise a labeled compound or agent capable of detecting GAVE6 protein or mRNA in a biological sample and means for determining the amount of GAVE6 in the sample (e.g., an anti-GAVE6 antibody or an oligonucleotide probe that binds to DNA encoding GAVE6, e.g., SEQ ID NO: 1). Kits also can be used to yield results indicating whether the tested subject is suffering from or is at risk of developing a disorder associated with aberrant expression of GAVE6, if the amount of GAVE6 protein or mRNA is above or below a normal level.

For antibody-based kits, the kit can comprise, for example: (1) a first antibody (e.g., attached to a solid support) that binds to GAVE6 protein; and, optionally, (2) a second, different antibody that binds to GAVE6 protein or to the first antibody and is conjugated to a detectable agent. If the second antibody is not present, then either the first antibody can be detectably labeled, or alternatively, another molecule that binds the first antibody can be detectably labeled. In any event, a labeled binding moiety is included to serve as the detectable reporter molecule, as known in the art.

For oligonucleotide-based kits, a kit of the present invention can comprise, for example: (1) an oligonucleotide, e.g., a detectably-labeled oligonucleotide, that hybridizes to a GAVE6 nucleic acid sequence or (2) a pair of primers useful for amplifying a GAVE6 nucleic acid molecule.

The kit also can comprise, e.g., a buffering agent, a preservative or a protein stabilizing agent. The kit also can comprise components necessary for detecting the detectable agent (e.g., an enzyme or a substrate). Furthermore, the kit may also contain a control sample or series of control samples that can be assayed and compared to the test sample. Each component of the kit is usually enclosed within an individual container, and all of the various containers are within a single package. Instructions for observing whether the tested subject is suffering from or is at risk of developing a disorder associated with aberrant expression of GAVE6 may also be enclosed.

2. Prognostic Assays

The methods described herein furthermore can be utilized as diagnostic or prognostic assays to identify subjects having or are at risk of developing a disease or disorder associated with aberrant GAVE6 expression or activity. For example, the assays described herein, such as the preceding diagnostic assays or the following assays, can be utilized to identify a subject having, or is at risk of developing, a disorder associated with GAVE6 protein, nucleic acid expression or activity. For example, recent contact with bacteria or inflammation associated with asthma, chronic obstructive pulmonary disease and rheumatoid arthritis are amenable for assay. Alternatively, the prognostic assays can be utilized to identify a subject having or is at risk for developing such a disease or disorder.

Thus, the instant invention provides a method in which a test sample is obtained from a subject and GAVE6 protein or nucleic acid (e.g., mRNA or genomic DNA) is detected. The presence of GAVE6 protein or nucleic acid is diagnostic of a subject having, or is at risk of developing, a disease or disorder associated with aberrant GAVE6 expression or activity. As used herein, a “test sample” refers to a biological sample obtained from a subject of interest. For example, a test sample can be a biological fluid (e.g., serum), cell sample or tissue.

Furthermore, the prognostic assays described herein can be used to determine whether a subject can be administered an agent (e.g., an agonist, antagonist, peptidomimetic, protein, peptide, nucleic acid, small molecule or other drug candidate) to treat a disease or disorder associated with aberrant GAVE6 expression or activity. For example, such methods can be used to determine whether a subject can be treated effectively with a specific agent or class of agents (e.g., agents of a type that decrease GAVE6 activity). Thus, the instant invention provides methods for determining whether a subject can be treated effectively with an agent for a disorder associated with aberrant GAVE6 expression or activity in which a test sample is obtained and GAVE6 protein or nucleic acid is detected (e.g., wherein the presence of GAVE6 protein or nucleic acid is diagnostic of a subject that can be administered the agent to treat a disorder associated with aberrant GAVE6 expression or activity).

The methods of the invention also can be used to detect genetic lesions or mutations in a GAVE6 gene, and thereby determine whether a subject with the lesioned gene is at risk for a disorder characterized by aberrant cell proliferation and/or differentiation. In preferred embodiments, the methods include detecting, in a sample of cells from the subject, the presence or absence of a genetic lesion or mutation characterized by at least one of an alteration affecting the integrity of a gene encoding a GAVE6-protein or the mis-expression of the GAVE6 gene. For example, such genetic lesions or mutations can be detected by ascertaining the existence of at least one of: 1) a deletion of one or more nucleotides from a GAVE6 gene; 2) an addition of one or more nucleotides to a GAVE6 gene; 3) a substitution of one or more nucleotides of a GAVE6 gene; 4) a chromosomal rearrangement involving a GAVE6 gene; 5) an alteration in the level of a messenger RNA transcript of a GAVE6 gene; 6) an aberrant modification of a GAVE6 gene, such as of the methylation pattern of the genomic DNA; 7) a non-wild type level of a GAVE6 protein; 8) an allelic loss of a GAVE6 gene; and 9) an inappropriate post-translational modification of a GAVE6 protein. As described herein, there are a large number of assay techniques known in the art that can be used for detecting lesions in a GAVE6 gene. A preferred biological sample is a peripheral blood leukocyte sample isolated by conventional means from a subject.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al., Science (1988) 241:1077-1080; and Nakazawa et al., Proc Natl Acad Sci USA (1994) 91:360-364), the latter of which can be particularly useful for detecting point mutations in the GAVE6 gene (see, e.g., Abravaya et al., Nucleic Acids Res (1995) 23:675-682). The method can include the steps of collecting a sample of cells from a patient, isolating nucleic acid (e.g., genomic, mRNA or both) from the cells of the sample, contacting the nucleic acid sample with one or more primers that specifically hybridize to a GAVE6 gene under conditions such that hybridization and amplification of the GAVE6 gene (if present) occurs and detecting the presence or absence of an amplification product or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self-sustained sequence replication (Guatelli et al., Proc Natl Acad Sci USA (1990) 87:1874-1878), transcriptional amplification system (Kwoh et al., Proc Natl Acad Sci USA (1989) 86:1173-1177), Q-β replicase (Lizardi et al., Bio/Technology (1988) 6:1197) or any other nucleic acid amplification method, followed by the detection of the amplified molecules using techniques well known to those of skill in the art. The detection schemes are especially useful for the detection of nucleic acid molecules if such molecules are present in very low numbers.

In an alternative embodiment, mutations in a GAVE6 gene from a sample cell can be identified by alterations in restriction enzyme cleavage patterns. For example, sample and control DNA is isolated, amplified (optionally), digested with one or more restriction endonucleases and fragment length sizes are determined by gel electrophoresis and compared. Differences in fragment length sizes between sample and control DNA indicate mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations in GAVE6 can be identified by hybridizing a sample and control nucleic acids, e.g., DNA or RNA, to high density arrays containing hundreds or thousands of oligonucleotides probes (Cronin et al., Human Mutation (1996) 7:244-255; Kozal et al., Nature Medicine (1996) 2:753-759). For example, genetic mutations in GAVE6 can be identified in two-dimensional arrays containing light-generated DNA probes as described in Cronin et al., supra. Briefly, a first hybridization array of probes can be used to scan through long stretches of DNA in a sample and control to identify base changes between the sequences by generating linear arrays of sequential overlapping probes. That step allows the identification of point mutations. The step is followed by a second hybridization array that allows the characterization of specific mutations by using smaller, specialized probe arrays complementary to all variants or mutations detected. Each mutation array is composed of parallel probe sets, one complementary to the wild type gene and the other complementary to the mutant gene.

In yet another embodiment, any of a variety of sequencing reactions known in the art can be used to directly sequence the GAVE6 gene and detect mutations by comparing the sequence of the sample GAVE6 with the corresponding wild type (control) sequence. Examples of sequencing reactions include those based on techniques developed by Maxam & Gilbert (Proc Natl Acad Sci USA (1977) 74:560) or Sanger (Proc Natl Acad Sci USA (1977) 74:5463). It also is contemplated that any of a variety of automated sequencing procedures can be utilized when performing the diagnostic assays (Bio/Techniques (1995) 19:448), including sequencing by mass spectrometry (see, e.g., PCT Publication No. WO 94/16101; Cohen et al., Adv Chromatogr (1996) 36:127-162; and Griffin et al., Appl Biochem Biotechnol (1993) 38:147-159).

Other methods for detecting mutations in the GAVE6 gene include methods in which protection from cleavage agents is used to detect mismatched bases in RNA/RNA or RNA/DNA heteroduplexes (Myers et al., Science (1985) 230:1242). In general, the technique of “mismatch cleavage” entails providing heteroduplexes formed by hybridizing (labeled) RNA or DNA containing the wild type GAVE6 sequence with potentially mutant RNA or DNA obtained from a tissue sample. The double-stranded duplexes are treated with an agent that cleaves single-stranded regions of the duplex such as that will exist due to base pair mismatches between the control and sample strands. RNA/DNA duplexes can be treated with RNase to digest mismatched regions and DNA/DNA hybrids can be treated with SI nuclease to digest mismatched regions. In other embodiments, either DNA/DNA or RNA/DNA duplexes can be treated with hydroxylamine or osmium tetroxide and with piperidine to digest mismatched regions. After digestion of the mismatched regions, the resulting material then is separated by size on denaturing polyacrylamide gels to determine the site of mutation. See, e.g., Cotton et al., Proc Natl Acad Sci USA (1988) 85:4397; Saleeba et al., Methods Enzymol (1992) 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in GAVE6 cDNAs obtained from samples of cells. For example, the mutY enzyme of E. coli cleaves A at G/A mismatches and the thymidine DNA glycosylase from HeLa cells cleaves T at G/T mismatches (Hsu et al., Carcinogenesis (1994) 15:1657-1662). According to an exemplary embodiment, a probe based on a GAVE6 sequence, e.g., a wild type GAVE6 sequence, is hybridized to a cDNA or other DNA product from a test cell(s). The duplex is treated with a DNA mismatch repair enzyme and the cleavage products, if any, can be detected in electrophoresis protocols or the like, see, e.g., U.S. Pat. No. 5,459,039.

In other embodiments, alterations in electrophoretic mobility will be used to identify mutations in GAVE6 genes. For example, single-strand conformation polymorphism (SSCP) may be used to detect differences in electrophoretic mobility between mutant and wild type nucleic acids (Orita et al., Proc Natl Acad Sci USA (1989) 86:2766; see also Cotton, Mutat Res (1993) 285:125-144; Hayashi, Genet Anal Tech Appl (1992) 9:73-79). Single-stranded DNA fragments of sample and control GAVE6 nucleic acids will be denatured and allowed to renature. The secondary structure of single-stranded nucleic acids varies according to sequence and the resulting alteration in electrophoretic mobility enables the detection of even a single base change. The DNA fragments may be labeled or detected with labeled probes. The sensitivity of the assay may be enhanced by using RNA (rather than DNA) because the secondary structure of RNA is more sensitive to a change in sequence. In a preferred embodiment, the subject method utilizes heteroduplex analysis to separate double-stranded heteroduplex molecules on the basis of changes in electrophoretic mobility (Keen et al., Trends Genet (1991) 7:5).

In yet another embodiment, the movement of mutant or wild type fragments in polyacrylamide gels containing a gradient of denaturant is assayed using denaturing gradient gel electrophoresis (DGGE) (Myers et al., Nature (1985) 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum et al., Biophys Chem (1987) 265:12753).

Examples of other techniques for detecting point mutations include, but are not limited to, selective oligonucleotide hybridization, selective amplification or selective primer extension. For example, oligonucleotide primers may be prepared in that the known mutation is placed centrally and then hybridized to target DNA under conditions that permit hybridization only if a perfect match is found (Saiki et al., Nature (1986) 324:163); Saiki et al., Proc Natl Acad Sci USA (1989) 86:6230). Such allele-specific oligonucleotides are hybridized to PCR-amplified target DNA or a number of different mutations when the oligonucleotides are attached to the hybridizing membrane and hybridized with labeled target DNA.

Alternatively, allele-specific amplification technology that depends on selective PCR amplification may be used in conjunction with the instant invention. Oligonucleotides used as primers for specific amplification may carry the mutation of interest in the center of the molecule (so that amplification depends on differential hybridization) (Gibbs et al., Nucleic Acids Res (1989) 17:2437-2448) or at the extreme 3′ end of one primer where, under appropriate conditions, mismatch can prevent or reduce polymerase extension (Prossner, Tibtech (1993) 11:238). In addition, it may be desirable to introduce a novel restriction site in the region of the mutation to create cleavage-based detection (Gasparini et al., Mol Cell Probes (1992) 6:1). It is anticipated that in certain embodiments amplification also may be performed using Taq ligase for amplification (Barany, Proc Natl Acad Sci USA (1991) 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein. The method and kit may be used conveniently, e.g., in clinical settings, to diagnose patients exhibiting symptoms or family history of a disease or illness involving a GAVE6 gene.

Furthermore, any cell type or tissue where GAVE6 is expressed may be utilized in the prognostic assays described herein.

3. Pharmacogenomics

Agents or modulators that have a stimulatory or inhibitory effect on GAVE6 activity (e.g., GAVE6 gene expression) as identified by a screening assay described herein can be administered to individuals to treat (prophylactically or therapeutically) disorders (e.g., inflammation associated with asthma, chronic obstructive pulmonary disease and rheumatoid arthritis) associated with GAVE6 activity. In conjunction with such treatment, the pharmacogenomics (i.e., the study of the relationship between the genotype of an individual and the response of the individual to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the genotype of the individual. Such pharmacogenomics further can be used to determine appropriate dosages and therapeutic regimens. Accordingly, the activity of GAVE6 protein, expression of GAVE6 nucleic acid or mutation content of GAVE6 genes in an individual can be determined thereby to select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder, Clin Chem (1997) 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism.” The pharmacogenetic conditions can occur either as rare defects or as polymorphisms. For example, glucose-6-phosphate dehydrogenase deficiency (G6PD) is a common inherited enzymopathy in that the main clinical complication is hemolysis after ingestion of oxidant drugs (anti-malarials, sulfonamides, analgesics or nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes, CYP2D6 and CYP2Cl 9) has provided an explanation as to why some patients do not obtain the expected drug effects or show exaggerated drug response and serious toxicity after taking the standard and safe dose of a drug. The polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, all which lead to the absence of functional CYP2D6. Poor metabolizers of CYP2D6 and CYP2Cl 9 quite frequently experience exaggerated drug response and side effects when standard doses are received. If a metabolite is the active therapeutic moiety, a PM will show no therapeutic response, as demonstrated for the analgesic effect of codeine mediated by the CYP2D6-formed metabolite, morphine. The other extreme is the so-called ultra-rapid metabolizers who do not respond to standard doses. Recently, the molecular basis of ultra-rapid metabolism has been identified to be due to CYP2D6 gene amplification.

Thus, the activity of GAVE6 protein, expression of GAVE6 nucleic acid or mutation content of GAVE6 genes in an individual can be determined to select thereby appropriate agent(s) for therapeutic or prophylactic treatment of the individual. In addition, pharmacogenetic studies can be used to apply genotyping of polymorphic alleles encoding drug-metabolizing enzymes to the identification of the drug responsiveness phenotype of an individual. That knowledge, when applied to dosing or drug selection, can avoid adverse reactions or therapeutic failure and thus enhance therapeutic or prophylactic efficiency when treating a subject with a GAVE6 modulator, such as a modulator identified by one of the exemplary screening assays described herein.

4. Monitoring of Effects During Clinical Trials

Monitoring the influence of agents (e.g., drugs or compounds) on the expression or activity of GAVE6 (e.g., the ability to modulate aberrant cell proliferation and/or differentiation) can be applied not only in basic drug screening, but also in clinical trials. For example, the effectiveness of an agent, as determined by a screening assay as described herein, to increase GAVE6 gene expression, protein levels or protein activity, can be monitored in clinical trials of subjects exhibiting decreased GAVE6 gene expression, protein levels or protein activity. Alternatively, the effectiveness of an agent, as determined by a screening assay, to decrease GAVE6 gene expression, protein levels or protein activity, can be monitored in clinical trials of subjects exhibiting increased GAVE6 gene expression, protein levels or protein activity. In such clinical trials, GAVE6 expression or activity and preferably, that of other genes that have been implicated in, for example, a cellular proliferation disorder, can be used as a marker of the immune responsiveness of a particular cell. For example, and not by way of limitation, genes, including GAVE6, that are modulated in cells by treatment with an agent (e.g., compound, drug or small molecule) that modulates GAVE6 activity (e.g., as identified in a screening assay described herein) can be identified. Thus, to study the effect of agents on cellular proliferation disorders, for example, in a clinical trial, cells can be isolated and RNA prepared and analyzed for the levels of expression of GAVE6 and other genes implicated in the disorder. The levels of gene expression (i.e., a gene expression pattern) can be quantified by Northern blot analysis or RT-PCR, as described herein, or alternatively by measuring the amount of protein produced by one of the methods as described herein or by measuring the levels of activity of GAVE6 or other genes. In that way, the gene expression pattern can serve as a marker, indicative of the physiological response of the cells to the agent. Accordingly, the response state may be determined before and at various points during treatment of the individual with the agent.

In a particular embodiment, the instant invention provides a method for monitoring the effectiveness of treatment of a subject with an agent (e.g., an agonist, antagonist, peptidomimetic, protein, peptide, nucleic acid, small molecule or other drug candidate identified by the screening assays described herein) comprising the steps of (i) obtaining a pre-administration sample from a subject prior to administration of the agent; (ii) detecting the level of expression of a GAVE6 protein, mRNA or genomic DNA in the preadministration sample; (iii) obtaining one or more post-administration samples from the subject; (iv) detecting the level of expression or activity of the GAVE6 protein, mRNA or genomic DNA in the post-administration samples; (v) comparing the level of expression or activity of the GAVE6 protein, mRNA or genomic DNA in the pre-administration sample with the GAVE6 protein, mRNA or genomic DNA in the post-administration sample or samples; and (vi) altering the administration of the agent to the subject accordingly. For example, increased administration of the agent may be desirable to increase the expression or activity of GAVE6 to higher levels than detected, i.e., to increase the effectiveness of the agent. Alternatively, decreased administration of the agent may be desirable to decrease expression or activity of GAVE6 to lower levels than detected, i.e., to decrease the effectiveness of the agent.

D. Methods of Treatment

The instant invention provides for both prophylactic and therapeutic methods of treating a subject at risk of (or susceptible to) a disorder or having a disorder associated with aberrant GAVE6 expression or activity. Such disorders include, but are not limited to, for example, inflammatory disorders such as asthma, chronic obstructive pulmonary disease and rheumatoid arthritis.

I. Prophylactic Methods

In one aspect, the invention provides a method for preventing in a subject, a disease or condition associated with an aberrant GAVE6 expression or activity, by administering to the subject an agent that modulates GAVE6 expression or at least one GAVE6 activity. Subjects at risk for a disease that is caused by or contributed to by aberrant GAVE6 expression or activity can be identified by, for example, any or a combination of diagnostic or prognostic assays as described herein. Administration of a prophylactic agent can occur prior to the manifestation of symptoms characteristic of the GAVE6 aberrancy, such that a disease or disorder is prevented or, alternatively, delayed in progression. Depending on the type of GAVE6 aberrancy, for example, a GAVE6 agonist or GAVE6 antagonist agent can be used for treating the subject. The appropriate agent can be determined based on screening assays described herein.

2. Therapeutic Methods

Another aspect of the invention pertains to methods of modulating GAVE6 expression or activity for therapeutic purposes. The modulatory method of the invention involves contacting a cell with an agent that modulates one or more of the activities of GAVE6 protein activity associated with the cell. An agent that modulates GAVE6 protein activity can be an agent as described herein, such as a nucleic acid or a protein, a naturally-occurring cognate ligand of a GAVE6 protein, a peptide, a GAVE6 peptidomimetic or other small molecule. In one embodiment, the agent stimulates one or more of the biological activities of GAVE6 protein. Examples of such stimulatory agents include active GAVE6 protein and a nucleic acid molecule encoding GAVE6 that has been introduced into the cell. In another embodiment, the agent inhibits one or more of the biological activities of GAVE6 protein. Examples of such inhibitory agents include antisense GAVE6 nucleic acid molecules and anti-GAVE6 antibodies. The modulatory methods can be performed in vitro (e.g., by culturing the cell with the agent) or, alternatively, in vivo (e.g., by administering the agent to a subject). As such, the instant invention provides methods of treating an individual afflicted with a disease or disorder characterized by aberrant expression or activity of a GAVE6 protein or nucleic acid molecule. In one embodiment, the method involves administering an agent (e.g., an agent identified by a screening assay described herein) or combination of agents that modulates (e.g., upregulates or downregulates) GAVE6 expression or activity. In another embodiment, the method involves administering a GAVE6 protein or nucleic acid molecule as therapy to compensate for reduced or aberrant GAVE6 expression or activity.

Stimulation of GAVE6 activity is desirable in situations in which GAVE6 is downregulated abnormally and/or in which increased GAVE6 activity is likely to have a beneficial effect. Conversely, inhibition of GAVE6 activity is desirable in situations in which GAVE6 is upregulated abnormally and/or in which decreased GAVE6 activity is likely to have a beneficial effect.

The present invention may be better understood by reference to the following non-limiting Example, which is provided as exemplary of the invention. The following Example is presented in order to more fully illustrate the preferred embodiments of the invention. It should in no way be construed, however, as limiting the broad scope of the invention.

### EXAMPLE

Materials and Methods

Identification of GAVE6. Homology searching against human genome sequence database HTG (NCBI/NIH) using various GPCR as queries was carried out using FASTA algorithm (Wisconsin GCG Package Version 10.1). Genomic DNA sequences that returned having statistically significant were translated into three forward frames for BLASTp searching of protein databases. A genomic DNA sequence AC013396 was identified to contain a putative GPCR sequence and was then named as GAVE6. Chromosome location of GAVE6 is mapped at 2p22.1.

Cloning of genomic DNA encoding GAVE6. Primers specific for the 5′ and 3′ sequences of the predicted GAVE6 were designed. Forward primer HP157, CAG CCC ATG GAA CTT CAT AAC CTG (SEQ ID NO: 5), and reverse primer HP158, CTG GCC CTC AGC CCT GGG AGG AG (SEQ ID NO: 6), were used to amplify GAVE6 genomic DNA by polymerase chain reaction (PCR) using human genomic DNA as template. PCR conditions were as follows: denaturation at 94° C. for 30 s, annealing at 55 ° C. for 30 s, and extension at 72° C. for 1 min, for 35 cycles, followed by a 5-min extension at 72 ° C. Amplified DNA fragment was cloned into the pCRII-TOPO vector from Invitrogen. The cloned DNA insert was verified by DNA sequencing. All the PCR amplifications were done in DNA Engine Tetrad (M J Research, model PTC-225).

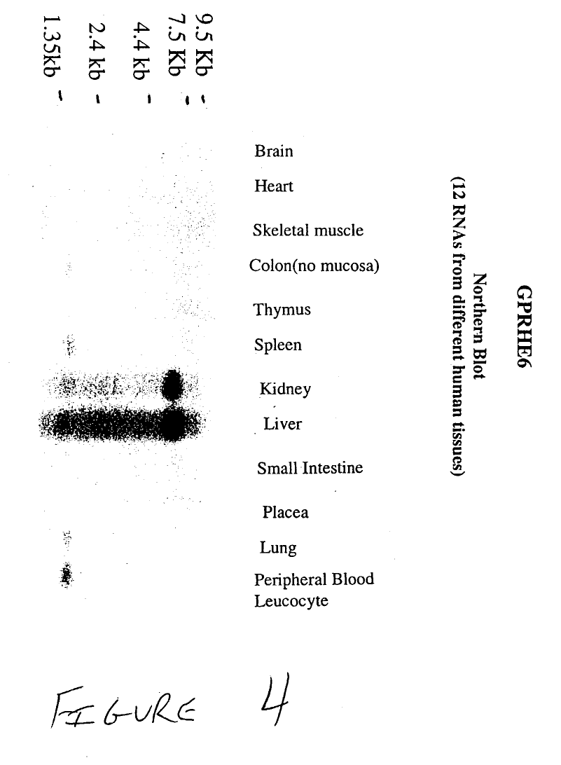

Northern blot analyses. Human multiple tissue Northern blots from Clontech were hybridized according to the manufacturer's instructions with [α³²P]dCTP labeled full-length open reading frame DNA fragment. Hybridized blots were washed with 2×SSPE and 0.1% SDS at 50° C. for 30 min and with 0.1×SSPE and 0.1%SDS at 50° C. for 1 hour. The blots were then exposed to X-ray film at −70 ° C. in the presence of an intensifying screen. The results of this Northern blot analysis are shown in FIG. 4.

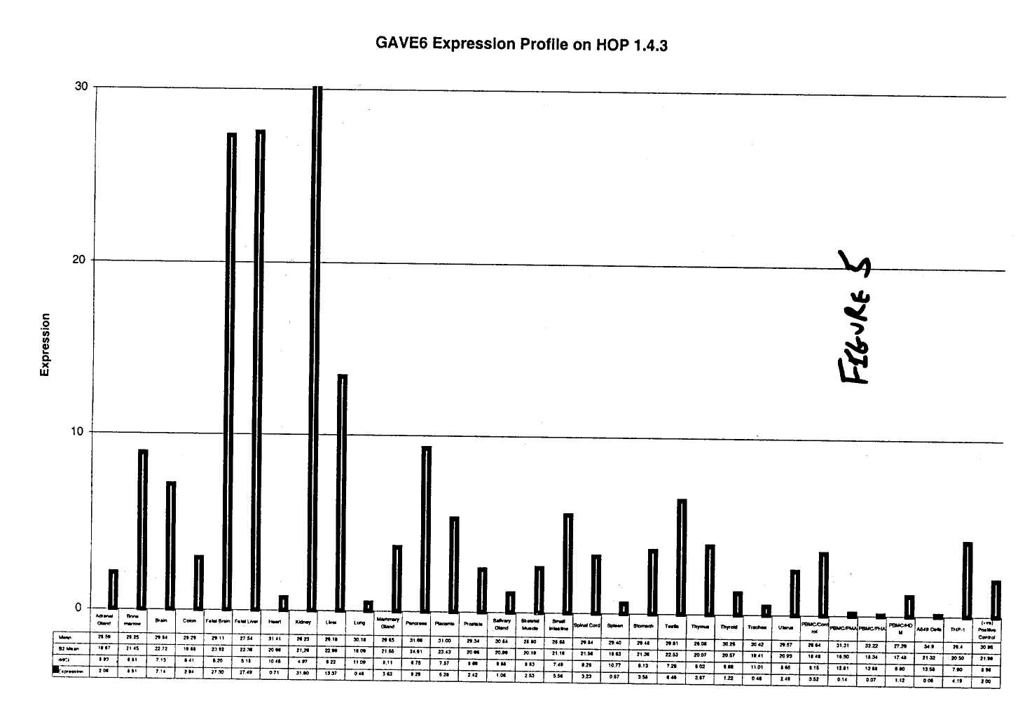

Taqman analysis. Total RNA from human tissues was purchased from Clontech. Prior to cDNA generation, total RNA was subjected to DNAse1 treatment to avoid potential genomic DNA contamination. In brief, Total RNA was mixed with 5 ul of 10× DNAseI buffer (20 mM Hepes pH 7.5; 10 mM CaCl₂; 10 mM MgCl₂; 11 mM DTT and 50% (v/v) glycerol) (Ambion), RNAse Inhibitor and 1 ul of DNAseI RNase free (2 U/ul; Ambion) in a final volume of 50 ul at 37C. for one hour. Following a phenol precipitation step, cDNA synthesis was performed using Superscript choice system as described by Life Technologies. Taqman primer/probes were designed using the Primer Express 1.0 software (ABI). The TaqMan forward primer of Gave 6: 5′ GCT GCC TGC AAA GTC AAC CT 3′ (SEQ ID NO: 7), the reverse primer: 5′ TGG CTG TGA GGA AGA CAA CG 3′ (SEQ ID NO: 8) and the Taqman probe sequence: 5° FAM-CCA CCA ACC GCA CGG CAA-TAMRA 3′ (SEQ ID NO: 9). Fam is used as a reporter dye and Tamra as a quencher. Taqman probe was custom synthesized by Operon Technologies. Taqman reactions was performed in a 96-well plate MicroAmp optical tube (PE) in a final volume of 50 ul containing: 25 ul Taqman PCR Mixture (Perkin Elmer); 1 ul Forward Primer for a final concentration of 900 nM; 1 ul Reverse Primer for a final concentration of 900 nM and 1 ul Taqman probe for a final concentration of 200 nM; 5 ul of cDNA template (calculated concentration of 10 ng/ul) and 17 ul of water. Taqman PCR condition was performed as described by PE Applied Biosystem. Human Beta actin primer probes (designed and purchased from PE applied Biosystem) was used as internal control. For each tissue, Taqman reactions were performed in duplicate for both target gene and internal control. In addition, a standard curve is generated for human beta actin in total brain cDNA using increasing amount of template in duplicate. This allows us to obtain relative number of amplicon amplified. Expression of target gene is expressed relative to brain cDNA as relative fold expression. The data obtained from the Taqman analysis are set forth below in Table 1 and graphically in FIG. 5.

TABLE 1

| Tissue Type | Mean | β 2 Mean | ∂∂ Ct | Expression |
|---|---|---|---|---|
| Adrenal Gland | 28.59 | 19.67 | 8.93 | 2.06 |
| Bone marrow | 28.25 | 21.45 | 6.81 | 8.91 |
| Brain | 29.84 | 22.72 | 7.13 | 7.14 |
| Colon | 28.29 | 19.88 | 8.41 | 2.94 |
| Fetal Brain | 29.11 | 23.92 | 5.20 | 27.30 |
| Fetal Liver | 27.54 | 22.36 | 5.18 | 27.49 |
| Heart | 31.41 | 20.96 | 10.45 | 0.71 |
| Kidney | 26.23 | 21.26 | 4.97 | 31.80 |
| Liver | 29.18 | 22.96 | 6.22 | 13.37 |
| Lung | 30.18 | 19.09 | 11.09 | 0.46 |
| Mammary Gland | 29.65 | 21.55 | 8.11 | 3.63 |
| Pancreas | 31.66 | 24.91 | 6.75 | 9.29 |
| Placenta | 31.00 | 23.43 | 7.57 | 5.28 |
| Prostate | 29.34 | 20.66 | 8.69 | 2.42 |
| Salivary Gland | 30.84 | 20.96 | 9.88 | 1.06 |
| Skeletal Muscle | 28.80 | 20.18 | 8.63 | 2.53 |
| Small Intestine | 28.68 | 21.19 | 7.49 | 5.56 |
| Spinal Cord | 29.84 | 21.56 | 8.28 | 3.23 |
| Spleen | 29.40 | 18.63 | 10.77 | 0.57 |
| Stomach | 29.48 | 21.36 | 8.13 | 3.58 |
| Testis | 29.81 | 22.53 | 7.28 | 6.46 |
| Thymus | 28.08 | 20.07 | 8.02 | 3.87 |
| Thyroid | 30.25 | 20.57 | 9.68 | 1.22 |
| Trachea | 30.42 | 19.41 | 11.01 | 0.48 |
| Uterus | 29.57 | 20.93 | 8.65 | 2.49 |
| PBMC/Control | 26.64 | 18.48 | 8.15 | 3.52 |
| PBMC/PMA | 31.31 | 18.50 | 12.81 | 0.14 |
| PBMC/PHA | 32.22 | 18.34 | 13.88 | 0.07 |
| PBMC/HDM | 27.29 | 17.48 | 9.80 | 1.12 |
| A549 Cells | 34.9 | 21.32 | 13.58 | 0.08 |
| THP-1 | 28.4 | 20.50 | 7.90 | 4.19 |
| (+ve) Positive Control | 30.95 | 21.98 | 8.96 | 2.00 |

PCR screening of cDNA library. PCR primers specific to the GAVE6 coding region: 5′TTC CTC CTG ATC AGC AAC CT 3′ (SEQ ID NO: 10), and 5′TTG GTG GAC AGC ATG AAG AG 3′ (SEQ ID NO: 11) were used to screen pooled human spleen, brain, kidney, activated T cells, and lung cDNA libraries. PCR screenings were done in 96-well plates using the following PCR protocol: 94° C, hold for 3 min; 40 cycles of 94° C. for 30 second, 52° C. for 30 second, and 68° C. for 45 second. Positive subpools were subsequently diluted for further round PCR screening. A limited number of colonies from positive subpools were plated on agar plates and positive plasmids were verified by PCR and were subjected for DNA sequencing analysis.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

What is claimed is:

1. An isolated nucleic acid molecule comprising the DNA sequence of FIG. 1 (SEQ ID NO: 1).

2. An isolated nucleic acid molecule hybridizable to said isolated nucleic acid molecule of claim 1, or a hybridization probe that is complementary to said isolated nucleic acid molecule of claim 1, under stringent hybridization conditions.

3. The isolated nucleic acid molecule of either of claims 1 or 2 which encodes a polypeptide having an amino acid sequence of FIG. 2 (SEQ ID NO: 2).

4. The isolated nucleic acid molecule of claim 2, which encodes a polypeptide having an amino acid sequence that is at least 30% identical to said amino acid sequence of SEQ ID NO: 2.

5. The isolated nucleic acid molecule of either of claims 1 or 2, which is detectably labeled.

6. The detectably labeled isolated nucleic acid molecule of claim 5, wherein said detectable label comprises an enzyme, a radioactive isotope, or a chemical which fluoresces.

7. A purified polypeptide comprising the amino acid sequence of FIG. 2 (SEQ ID NO: 2).

8. An isolated nucleic acid molecule which encodes said purified polypeptide of claim 7.

9. The purified polypeptide of claim 7 which is detectably labeled.

10. The purified polypeptide of claim 9, wherein said detectable label comprises an enzyme, a radio active isotope, or a chemical which fluoresces.

11. An antibody having said purified polypeptide of claim 7 as an immunogen.

12. The antibody of claim 11, wherein said antibody is selected from the group consisting of a monoclonal antibody, a polyclonal antibody, or a chimeric antibody.

13. The antibody of claim 11, which is detectably labeled.

14. The antibody of claim 13, wherein said detectable label comprises an enzyme, a radioactive isotope, or a chemical which fluoresces.

15. An expression vector comprising said isolated nucleic acid molecule of claim 1 operatively associated with an expression control element.

16. An expression vector comprising said isolated nucleic acid molecule of claim 2, operatively associated with an expression control element.

17. The expression vector of either of claims 15 or 16, wherein said expression control element is selected from the group consisting of a constitutive regulatory sequence, a cell-specific regulatory sequence, and an inducible regulatory sequence.

18. The expression vector of claim 17, wherein said expression control element is a promoter.

19. The expression vector of claim 18, wherein said promoter comprises an immediate early promoter of hCMV, an early promoter of SV40, an early promoter of adenovirus, an early promoter of vaccinia, an early promoter of polyoma, a late promoter of SV40, a late promoter of adenovirus, a late promoter of vaccinia, a late promoter of polyoma, a lac system, a trp system, a TAC system, a TRC system, a major operator and promoter region of phage lambda, a control region of fd coat protein, 3-phosphoglycerate kinase promoter, acid phosphatase promoter, or a promoter of yeast α mating factor.

20. A host cell transformed or transfected with the expression vector of either of claims 15 or 16.

21. The host cell of claim 20, wherein said host cell comprises a prokaryotic cell or eukaryotic cell.

22. The host cell of claim 21, wherein said host comprises E. coli, Pseudonomas, Bacillus, Strepomyces, yeast, CHO, R1.1, B-W, L-M, COS1, COS7, BSC1, BSC40, BMT10 or Sf9 cells.

23. A method for producing the isolated polypeptide of claim 7, comprising the steps of:
a) culturing a host cell of claim 20 under conditions that provide for expression of said isolated polypeptide; and
b) recovering said isolated polypeptide from said host, said culture, or a combination thereof.

24. A therapeutic method for modulating GAVE6 signaling activity or signal transduction in a patient in need of treatment comprising administering to said patient an agonist, an antagonist or an inverse agonist of GAVE6.

25. A method for identifying an agonist of GAVE6 comprising: contacting a potential agonist with a cell expressing GAVE6 and determining whether in the presence of said potential agonist the signaling activity of GAVE6 is increased relative to the activity of GAVE6 in the absence of said potential agonist.

26. A method for identifying an inverse agonist of GAVE6 comprising: contacting a potential inverse agonist with a cell expressing GAVE6 and determining whether in the presence of said potential inverse agonist, the activity of GAVE6 is decreased relative to the activity of GAVE6 in the absence of said potential inverse agonist, and is decreased in the presence of an endogenous ligand or agonist.

27. A method for identifying an antagonist of GAVE6 comprising: contacting a potential antagonist with a cell expressing GAVE6 and determining whether in the presence of said potential antagonist the signaling activity of GAVE6 is decreased relative to the activity of GAVE6 in the presence of an endogenous ligand or agonist.

28. A therapeutic composition comprising an agonist, an antagonist, or an inverse agonist of GAVE6 capable of modulating GAVE6 signaling activity or transduction.

29. A method for treating a disease comprising administering to a patient in need of treatment a therapeutic composition comprising an agonist, an antagonist or an inverse agonist of GAVE6 capable of modulating GAVE6 signaling activity or transduction.
